(19) United States
(12) Patent Application Publication
(10) Pub. No.: US 2024/0018508 A1
(43) Pub. Date: Jan. 18, 2024

(54) PHAGE CYCLISATION ASSAY

(71) Applicant: BicycleRD Limited, Cambridge (GB)

(72) Inventors: Michael SKYNNER, Cambridge (GB); James COOKE, Cambridge (GB); Emma CRAWLEY, Cambridge (GB); Liuhong CHEN, Cambridge (GB); Gemma MUDD, Cambridge (GB); Paul BESWICK, Cambridge (GB)

(21) Appl. No.: 17/766,167

(22) PCT Filed: Oct. 2, 2020
(86) PCT No.: PCT/EP2020/077739

§ 371 Date: Apr. 1, 2022

(30) Foreign Application Priority Data
Oct. 2, 2019 (GB) 1914233.0

(51) Int. Cl. C12N 15/10

(52) U.S. Cl. CPC C12N 15/1037

(57) ABSTRACT

The present invention relates to a method for determining an extent of cyclisation of a peptide ligand displayed on a genetic display system, wherein the peptide ligand comprises a polypeptide covalently linked to a molecular scaffold at two or more amino acid residues, comprising the steps of exposing the polypeptide displayed on the genetic display system to the molecular scaffold, wherein said polypeptide comprises two or more peptide reactive groups on said two or more amino acid residues which form covalent bonds with the molecular scaffold at two or more scaffold reactive groups, to give the peptide ligand; removing unreacted molecular scaffold from the genetic display system; exposing the peptide ligand displayed on the genetic display system to a first probe, wherein the first probe binds to a first unconjugated reactive group on the peptide ligand; and measuring the first unconjugated reactive group on the peptide ligand.

### BACKGROUND OF THE INVENTION

Cyclic peptides are able to bind with high affinity and target specificity to protein targets and hence are an attractive molecule class for the development of therapeutics. In fact, several cyclic peptides are successfully used in the clinic, as for example the antibacterial peptide vancomycin, the immunosuppressant drug cyclosporine or the anti-cancer drug ocreotide (Driggers, et al., Nat Rev Drug Discov 2008, 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu, B., et al., Science 330 (6007), 1066-71), a cyclic peptide with the Arg-Gly-Asp motif binding to integrin αVb3 (355 Å²) (Xiong, J. P., et al., Science 2002, 296 (5565), 151-5) or the cyclic peptide inhibitor upain-1 binding to urokinase-type plasminogen activator (603 Å²; Zhao, G., et al., J Struct Biol 2007, 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8, MMP-8) which lost its selectivity over other MMPs when its ring was opened (Cherney, R. J., et al., J Med Chem 1998, 41 (11), 1749-51). The favorable binding properties achieved through macrocyclization are even more pronounced in multicyclic peptides having more than one peptide ring as for example in vancomycin, nisin or actinomycin.

Different research teams have previously tethered polypeptides with cysteine residues to a synthetic molecular structure (Kemp, D. S. and McNamara, P. E., J. Org. Chem, 1985; Timmerman, P. et al., ChemBioChem, 2005). Meloen and co-workers had used tris(bromomethyl)benzene and related molecules for rapid and quantitative cyclisation of multiple peptide loops onto synthetic scaffolds for structural mimicry of protein surfaces (Timmerman, P. et al., ChemBioChem, 2005). Methods for the generation of candidate drug compounds wherein said compounds are generated by linking cysteine containing polypeptides to a molecular scaffold as for example tris(bromomethyl)benzene are disclosed in WO 2004/077062, WO 2006/078161 and WO 2018/197893.

WO 2004/077062 discloses a method of selecting a candidate drug compound. In particular, this document discloses various scaffold molecules comprising first and second reactive groups, and contacting said scaffold with a further molecule to form at least two linkages between the scaffold and the further molecule in a coupling reaction.

WO 2006/078161 discloses binding compounds, immunogenic compounds and peptidomimetics. This document discloses the artificial synthesis of various collections of peptides taken from existing proteins. These peptides are then combined with a constant synthetic peptide having some amino acid changes introduced in order to produce combinatorial libraries. By introducing this diversity via the chemical linkage to separate peptides featuring various amino acid changes, an increased opportunity to find the desired binding activity is provided. FIG. 1 of this document shows a schematic representation of the synthesis of various loop peptide constructs. The constructs disclosed in this document rely on —SH functionalised peptides, typically comprising cysteine residues, and heteroaromatic groups on the scaffold, typically comprising benzylic halogen substituents such as bis- or tris-bromophenylbenzene. Such groups react to form a thioether linkage between the peptide and the scaffold.

Heinis et al. developed a phage display-based combinatorial approach to generate and screen large libraries of bicyclic peptides to targets of interest (Heinis, et al., Nat Chem Biol 2009, 5 (7), 502-7; see also international patent application WO 2009/098450) (FIG. 1A). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule (tris-(bromomethyl)benzene). Bicyclic peptides isolated in selections for affinity to the human proteases cathepsin G and plasma Kallikrein (PK) had nanomolar inhibitory constants. The best inhibitor, PK15, inhibits human PK (hPK) with a Kᵢ of 3 nM. Similarities in the amino acid sequences of several isolated bicyclic peptides suggested that both peptide loops contribute to the binding. PK15 did not inhibit rat PK (81% sequence identity) nor the homologous human serine proteases factor XIa (hfXIa; 69% sequence identity) or thrombin (36% sequence identity) at the highest concentration tested (10 μM) (Heinis, et al., Nat Chem Biol 2009, 5 (7), 502-7). This finding suggested that the bicyclic inhibitor possesses high affinity for its target, and is highly specific. WO 2014/140342 further discloses an improved protocol for the production of bicyclic peptides displayed on phage.

Although the methods disclosed by Heinis et al. and WO 2014/140342 are effective for the modification of displayed peptide to produce bicyclic peptides, the extent of bicyclic peptide cyclisation cannot be precisely determined. Mass spectrometry (FIG. 1B) can be used for identifying the reaction product, but it is not a desirable tool for quantifying the yield of the cyclisation reaction, as a pure form of the resulting product is not always available for plotting a standard curve of signal intensity against concentration. Moreover, the quantification is more complicated when a library of peptide ligands (such as bicyclic peptides) is involved, as the extent of ionisation of a peptide or peptide ligand is dependent on the amino acid sequence. The signal intensity for two different peptides can be very different even if they have the same concentration.

In view of the above, a need exists for developing a simple and efficient assay for determining the extent of cyclisation of a peptide ligand displayed on a genetic display system. In particular, the assay should be applicable to a library of peptide ligands.

### SUMMARY OF THE INVENTION

The present invention provides a method for determining an extent of cyclisation of a peptide ligand displayed on a genetic display system, wherein the peptide ligand comprises a polypeptide covalently linked to a molecular scaffold at two or more amino acid residues, comprising the steps of:
(a) exposing the polypeptide displayed on the genetic display system to the molecular scaffold, wherein said polypeptide comprises two or more peptide reactive groups on said two or more amino acid residues which form covalent bonds with the molecular scaffold at two or more scaffold reactive groups, to give the peptide ligand;
(b) removing unreacted molecular scaffold from the genetic display system;
(c) exposing the peptide ligand displayed on the genetic display system to a first probe, wherein the first probe binds to a first unconjugated reactive group on the peptide ligand; and
(d) measuring the first unconjugated reactive group on the peptide ligand.

A precise determination of the extent of cyclisation is crucial for optimising the reaction conditions such as temperature, scaffold concentration, pH and reaction time. This is particularly important for the development of new molecular scaffolds. The present invention also allows comparison of cyclisation efficiency of different molecular scaffolds. Furthermore, the present invention assists the screening of specific clones with correct cyclisation, which could in turn facilitate the selection of a desired peptide ligand.

The amount, quantity and/or proportion of unconjugated reactive group can be measured based on the property of the probe. In certain embodiments, the probe can directly or indirectly generate a detectable and quantifiable signal so that the unconjugated reactive group can be measured. The signal, for example, can be fluorescence, luminescence, radioactive signal or any electromagnetic signal detectable by NMR, IR or Raman spectroscopy. In one embodiment, the probe comprises an enzyme or a catalyst which can catalyse a reaction to generate such a signal. In one embodiment, the probe can be activated or modified to generate such a signal.

In certain embodiments, the probe comprises or is linkable to a signalling group, wherein the signalling group is configured to produce a signal directly or indirectly to indicate the unconjugated reactive group on the peptide ligand.

In one embodiment, the probe comprises a signalling portion and a non-signalling portion. Preferably, the non-signalling portion comprises a probe reactive group which binds to a target.

In one embodiment, the probe comprises a signalling bead and a probe reactive group which binds to a target.

In one embodiment, the probe reactive group is linked to a polymer linker such as polyethylene glycol (PEG). In one embodiment, the probe comprises PEG2 or PEG3.

In certain embodiments, the first probe comprises a first probe reactive group which binds to a first unconjugated reactive group. In one embodiment, the first probe reactive group is identical to the peptide reactive group or the scaffold reactive group.

In certain embodiments, the first probe comprises or is linkable to a first signalling group, wherein the first signalling group produces a first signal directly or indirectly to indicate the first unconjugated reactive group on the peptide ligand.

In certain embodiments, the method further comprises exposing the peptide ligand displayed on the genetic display system to a second probe after step (c), wherein the second probe binds to the genetic display system, and comprises or is linkable to a second signalling group.

In one embodiment, the second signalling group is triggered by the first signal to produce a second signal.

In one embodiment, the second signalling group produces a second signal, the second signal triggering the first signalling group to produce the first signal.

In certain embodiments, the second probe comprises a second probe reactive group which binds to an antigen on the genetic display system. Preferably, the second probe reactive group is an antibody.

In one embodiment, the first (second) signalling group comprises a first photosensitiser configured to convert ambient oxygen molecules to singlet oxygen molecules, and the second (first) signalling group comprises a first chemiluminescent molecule configured to be excited by the singlet oxygen molecules. Preferably, the first chemiluminscent molecule is a thioxene derivative. Suitably, the second (first) signalling group further comprises a first fluorescent group, the first fluorescent group is configured to be excited by the chemiluminescence of the first chemiluminescent molecule.

In one embodiment, the first probe and the second probe form a donor and an acceptor of the Amplified Luminescent Proximity Homogeneous Assay screen (AlphaScreen) or AlphaLISA respectively. In one embodiment, the first probe and the second probe form an acceptor and a donor of the AlphaScreen or AlphaLISA respectively.

In certain embodiments, the first probe is fluorescent or linkable to a first fluorescent entity. In one embodiment, the first probe comprises a fluorescent portion and a non-fluorescent portion. Preferably, the non-fluorescent portion comprises the first probe reactive group. In one embodiment, the first probe comprises a fluorescent bead and a probe reactive group which binds to a target.

In certain embodiments, the second probe is fluorescent or linkable to a second fluorescent entity.

In one embodiment, the first probe or the first fluorescent entity has an emission spectrum overlapping with an absorption (or excitation) spectrum of the second probe or the second fluorescent entity. Preferably, the first probe (or fluorescent entity) and the second probe (or fluorescent entity) form the donor and the acceptor respectively for Forster resonance energy transfer (FRET).

In one embodiment, the second probe or the second fluorescent entity has an emission spectrum overlapping with an absorption (or excitation) spectrum of the first probe or the first fluorescent entity. Preferably, the first probe (or fluorescent entity) and the second probe (or fluorescent entity) form the acceptor and the donor respectively for FRET.

In certain embodiments, the first unconjugated reactive group is one of the two or more peptide reactive groups. Preferably, the first probe reactive group of the first probe binds to one of the two or more peptide reactive groups. Preferably, the first probe reactive group is identical to one of the two or more scaffold reactive groups which binds to the same target as that of the first probe. Preferably, the first probe reactive group comprises a maleimide group.

In one embodiment, the first probe binds to one of the two or more peptide reactive groups, the concentration of the first probe in step (c) is between 100 nM and 100 μM for a single clone of peptide ligand displayed on a genetic display system. Preferably, the concentration of the first probe is between 1 μM and 10 μM. Preferably, the concentration of the first probe is between 1 μM and 5 μM. Preferably, the concentration of the first probe is 2.5 μM. Suitably, the peptide ligand displayed on the genetic display system is exposed to the first probe for less than 5 hours, preferably less than 3 hours, more preferably less than 2 hours. Suitably, the peptide ligand displayed on the genetic display system is exposed to the first probe at room temperature. In one embodiment, the peptide ligand displayed on the genetic display system is exposed to the first probe for 2 hours at room temperature. Suitably, the single clone of peptide ligand displayed on a genetic display system is diluted for 10 times before exposing to the second probe after step (c).

In one embodiment, the first probe binds to one of the two or more peptide reactive groups, the concentration of the first probe in step (c) is between 1 nM and 10 μM for a library of peptide ligands displayed on a genetic display system. Preferably, the concentration of the first probe is between 10 nM and 1 μM. Preferably, the concentration of the first probe is between 50 nM and 500 nM. Preferably, the concentration of the first probe is between 50 nM and 150 nM. Preferably, the concentration of the first probe is 100 nM. Suitably, the peptide ligand displayed on the genetic display system is exposed to the first probe for less than 5 hours, preferably less than 3 hours, more preferably less than 2 hours. Suitably, the peptide ligand displayed on the genetic display system is exposed to the first probe at room temperature. In one embodiment, the peptide ligand displayed on the genetic display system is exposed to the first probe for 2 hours at room temperature. Suitably, the library of peptide ligands displayed on a genetic display system is diluted for 100 times before exposing to the second probe after step (c).

In one embodiment, the first unconjugated reactive group is one of the two or more scaffold reactive groups. Preferably, the first probe reactive group of the first probe binds to one of the two or more scaffold reactive groups. Preferably, the first probe reactive group is identical to one of the two or more peptide reactive groups which binds to the same target as that of the first probe. Preferably, the first probe reactive group comprises a thiol group.

Suitably, step (c) of the method further comprises treating the genetic display system with a reducing agent after exposing the genetic display system to the first probe. A suitable reducing agent is TCEP. Other reducing agents, such as DTT, can be used as set forth herein. Preferably, both the peptide reactive group and the first probe reactive group are a thiol group of cysteine. The reducing agent used is preferably included at a concentration of less than 500 mM, preferably less than 200 mM, advantageously less than 100 mM. For example, the reducing agent is present at a concentration of 10 mM or less, such as 1 mM. The addition of reducing agent prevents the formation of disulphide bonds between the peptide reactive group and the first probe reactive group. As the first probe reactive group is for targeting the scaffold reactive group, the addition of reducing agent can avoid false positive during the measurement of unconjugated scaffold reactive groups. Preferably, the genetic display system is neutralised after treating with the reducing agent.

In one embodiment, the first probe binds to one of the two or more scaffold reactive groups, the concentration of the first probe in step (c) is between 10 μM and 10 mM for a single clone of peptide ligand displayed on a genetic display system. Preferably, the concentration of the first probe is between 100 μM and 1 mM. Preferably, the concentration of the first probe is between 100 μM and 500 μM. Preferably, the concentration of the first probe is 320 μM. Suitably, the peptide ligand displayed on the genetic display system is exposed to the first probe for less than 4 hours, preferably less than 2 hours, more preferably less than 1 hour. Suitably, the peptide ligand displayed on the genetic display system is exposed to the first probe at room temperature. In one embodiment, the peptide ligand displayed on the genetic display system is exposed to the first probe for 1 hour at room temperature. Suitably, the single clone of peptide ligand displayed on a genetic display system is diluted for 100 times before exposing to the second probe after step (c).

In one embodiment, the first probe binds to one of the two or more scaffold reactive groups, the concentration of the first probe in step (c) is between 10 μM and 10 mM for a library of peptide ligands displayed on a genetic display system. Preferably, the concentration of the first probe is between 100 μM and 5 mM. Preferably, the concentration of the first probe is between 500 μM and 2.5 mM. Preferably, the concentration of the first probe is 1.28 mM. Suitably, the peptide ligand displayed on the genetic display system is exposed to the first probe for less than 4 hours, preferably less than 2 hours, more preferably less than 1 hour. Suitably, the peptide ligand displayed on the genetic display system is exposed to the first probe at room temperature. In one embodiment, the peptide ligand displayed on the genetic display system is exposed to the first probe for 1 hour at room temperature. Suitably, the library of peptide ligands displayed on a genetic display system is diluted for 10 times before exposing to the second probe after step (c).

In one embodiment, the first unconjugated reactive group is one of the two or more peptide reactive groups, wherein the method is further repeated by using a third probe in step (c), the third probe binds to a second unconjugated reactive group, wherein the second unconjugated reactive group is one of the two or more scaffold reactive groups. Preferably, the first probe reactive group of the first probe binds to one of the two or more peptide reactive groups. Preferably, the third probe comprises a third probe reactive group which binds to one of the two or more scaffold reactive groups. Suitably, the third probe reactive group is identical to one of the two or more peptide reactive groups which binds to the same target as that of the third probe. Preferably, the third probe reactive group comprises a thiol group. Suitably, step (c) of the second round of the method further comprises treating the genetic display system with a reducing agent after exposing the genetic display system to the third probe. A suitable reducing agent is TCEP. Other reducing agents, such as DTT, can be used as set forth herein. Preferably, both the peptide reactive group and the first probe reactive group are a thiol group of cysteine. The reducing agent used is preferably included at a concentration of less than 500 mM, preferably less than 200 mM, advantageously less than 100 mM. For example, the reducing agent is present at a concentration of 10 mM or less, such as 1 mM. Preferably, the genetic display system is neutralised after treating with the reducing agent.

In one embodiment, the first unconjugated reactive group is one of the two or more scaffold reactive groups, wherein the method is further repeated by using a third probe in step (c), the third probe binds to a second unconjugated reactive group, wherein the second unconjugated reactive group is one of the two or more peptide reactive groups. Preferably, the first probe reactive group of the first probe binds to one of the two or more scaffold reactive groups. Preferably, the third probe comprises a third probe reactive group which binds to one of the two or more peptide reactive groups. Suitably, the third probe reactive group is identical to one of the two or more scaffold reactive groups which binds to the same target as that of the third probe. Preferably, the third probe reactive group comprises a maleimide group.

In certain embodiments, the third probe comprises or is linkable to a third signalling group, wherein the third signalling group produces a third signal directly or indirectly to indicate the second unconjugated reactive group on the peptide ligand.

In certain embodiments, the method further comprises exposing the peptide ligand displayed on the genetic display system to a fourth probe after step (c), wherein the fourth probe binds to the genetic display system, and comprises or is linkable to a fourth signalling group.

In one embodiment, the fourth signalling group is triggered by the third signal to produce a fourth signal.

In one embodiment, the fourth signalling group produces a fourth signal, the fourth signal triggering the third signalling group to produce the third signal.

In certain embodiments, the fourth probe comprises a fourth probe reactive group which binds to an antigen on the genetic display system. Preferably, the fourth probe reactive group is an antibody. Suitably, the fourth probe is identical to the second probe.

In one embodiment, the third (fourth) signalling group comprises a second photosensitiser configured to convert ambient oxygen molecules to singlet oxygen molecules, and the fourth (third) signalling group comprises a second chemiluminescent molecule configured to be excited by the singlet oxygen molecules. Preferably, the second chemiluminscent molecule is a thioxene derivative. Suitably, the fourth (third) signalling group further comprises a second fluorescent group, the second fluorescent group is configured to be excited by the chemiluminescence of the second chemiluminescent molecule. Suitably, the second photosensitiser, the second chemiluminescent and the second fluorescent group are identical to the first photosensitiser, the first chemiluminescent and the first fluorescent group respectively.

In one embodiment, the third probe and the fourth probe form a donor and an acceptor of the AlphaScreen or AlphaLISA respectively. In one embodiment, the third probe and the fourth probe form an acceptor and a donor of the AlphaScreen or AlphaLISA respectively.

In certain embodiments, the third probe is fluorescent or linkable to a third fluorescent entity. In one embodiment, the third probe comprises a fluorescent portion and a non-fluorescent portion. Preferably, the non-fluorescent portion comprises the third probe reactive group. In one embodiment, the third probe comprises a fluorescent bead and a probe reactive group which binds to a target.

In certain embodiments, the fourth probe is fluorescent or linkable to a fourth fluorescent entity.

In one embodiment, the third probe or the third fluorescent entity has an emission spectrum overlapping with an absorption (or excitation) spectrum of the fourth probe or the fourth fluorescent entity. Preferably, the third probe (or fluorescent entity) and the fourth probe (or fluorescent entity) form the donor and the acceptor respectively for FRET.

In one embodiment, the fourth probe or the fourth fluorescent entity has an emission spectrum overlapping with an absorption (or excitation) spectrum of the third probe or the third fluorescent entity. Preferably, the third probe (or fluorescent entity) and the fourth probe (or fluorescent entity) form the acceptor and the donor respectively for FRET.

In one embodiment, the third probe binds to one of the two or more peptide reactive groups, the concentration of the third probe in step (c) is between 100 nM and 100 μM for a single clone of peptide ligand displayed on a genetic display system. Preferably, the concentration of the third probe is between 1 μM and 10 μM. Preferably, the concentration of the third probe is between 1 μM and 5 μM. Preferably, the concentration of the third probe is 2.5 μM. Suitably, the peptide ligand displayed on the genetic display system is exposed to the third probe for less than 5 hours, preferably less than 3 hours, more preferably less than 2 hours. Suitably, the peptide ligand displayed on the genetic display system is exposed to the third probe at room temperature. In one embodiment, the peptide ligand displayed on the genetic display system is exposed to the third probe for 2 hours at room temperature. Suitably, the single clone of peptide ligand displayed on a genetic display system is diluted for 10 times before exposing to the fourth probe after step (c).

In one embodiment, the third probe binds to one of the two or more peptide reactive groups, the concentration of the third probe in step (c) is between 1 nM and 10 μM for a library of peptide ligands displayed on a genetic display system. Preferably, the concentration of the third probe is between 10 nM and 1 μM. Preferably, the concentration of the third probe is between 50 nM and 500 nM. Preferably, the concentration of the third probe is between 50 nM and 150 nM. Preferably, the concentration of the third probe is 100 nM. Suitably, the peptide ligand displayed on the genetic display system is exposed to the third probe for less than 5 hours, preferably less than 3 hours, more preferably less than 2 hours. Suitably, the peptide ligand displayed on the genetic display system is exposed to the third probe at room temperature. In one embodiment, the peptide ligand displayed on the genetic display system is exposed to the third probe for 2 hours at room temperature. Suitably, the library of peptide ligands displayed on a genetic display system is diluted for 100 times before exposing to the fourth probe after step (c).

In one embodiment, the third probe binds to one of the two or more scaffold reactive groups, the concentration of the third probe in step (c) is between 10 μM and 10 mM for a single clone of peptide ligand displayed on a genetic display system. Preferably, the concentration of the third probe is between 100 μM and 1 mM. Preferably, the concentration of the third probe is between 100 μM and 500 μM. Preferably, the concentration of the third probe is 320 μM. Suitably, the peptide ligand displayed on the genetic display system is exposed to the third probe for less than 4 hours, preferably less than 2 hours, more preferably less than 1 hour. Suitably, the peptide ligand displayed on the genetic display system is exposed to the third probe at room temperature. In one embodiment, the peptide ligand displayed on the genetic display system is exposed to the third probe for 1 hour at room temperature. Suitably, the single clone of peptide ligand displayed on a genetic display system is diluted for 100 times before exposing to the fourth probe after step (c).

In one embodiment, the third probe binds to one of the two or more scaffold reactive groups, the concentration of the third probe in step (c) is between 10 μM and 10 mM for a library of peptide ligands displayed on a genetic display system. Preferably, the concentration of the third probe is between 100 μM and 5 mM. Preferably, the concentration of the third probe is between 500 μM and 2.5 mM. Preferably, the concentration of the third probe is 1.28 mM. Suitably, the peptide ligand displayed on the genetic display system is exposed to the third probe for less than 4 hours, preferably less than 2 hours, more preferably less than 1 hour. Suitably, the peptide ligand displayed on the genetic display system is exposed to the third probe at room temperature. In one embodiment, the peptide ligand displayed on the genetic display system is exposed to the third probe for 1 hour at room temperature. Suitably, the library of peptide ligands displayed on a genetic display system is diluted for 10 times before exposing to the fourth probe after step (c).

In certain embodiments, the two or more peptide reactive groups comprise cysteine residues.

In certain embodiments, the peptide ligand can be a single clone or a library of peptide ligands displayed on a genetic display system. A single clone of peptide ligand refers to peptide ligands having the same polypeptide sequence.

In certain embodiments, the genetic display system is selected from phage display, ribosome display, mRNA display, yeast display and bacterial display. In one embodiment, the genetic display system is phage display. Preferably, the polypeptide is displayed by fusion to the pIII protein of fd phage, such as fd-tet phage.

The library of peptide ligands has a complexity of at least 10⁴, 10⁵, 10⁶, 10⁷, 10⁸, 10⁹, 10¹⁰ or more peptide ligands. The library size can be at least 10 times the complexity, for example 10¹¹, 10¹², 10¹³ or more peptide ligands.

Libraries of peptide ligands can be prepared according to methods known in the art. For example, methods are described in Heinis et al., WO/2009/098450 and WO 2014/140342. The original method by Heinis et al. performed the conjugation of peptide and molecular scaffold (TBMB) in free solution. Phage, bearing peptides which were (or were not) conjugated to the TBMB scaffold were then isolated by centrifugation. Improved results have obtained by conjugating the phage to a solid phase purification resin, which can then be used to isolate the phage (refer to WO 2014/140342). For example, the resin can be isolated by centrifugation or retained in columns; in a preferred embodiment, the resin is magnetic and can be isolated by the application of a magnetic field. Either conjugation approach can be used with the present invention.

In certain embodiments, the genetic display system is combined with a purification resin before step (a) such that the genetic display system is bound to the purification resin.

The purification resin is useful as a solid phase for the purification of protein material. Many resins, such as ion-exchange resins including beads and chromatography materials are known in the art which are useful for this purpose.

In an advantageous embodiment, the resin is a magnetic resin, which allows magnetic separation of the polypeptides bound to the genetic display system.

Preferably, the bound genetic display system is further treated with a reducing agent before step (a). A suitable reducing agent is TCEP. Other reducing agents, such as DTT, can be used as set forth herein. The reducing agent used is preferably included at a concentration of less than 500 mM, preferably less than 200 mM, advantageously less than 100 mM. For example, the reducing agent is present at a concentration of 10 mM or less, such as 1 mM.

Preferably, the bound genetic display system is washed before addition of the molecular scaffold. Washing can be performed, for example, with a solution of a reducing agent. Advantageously, the reducing agent used in the washing step is less powerful or more dilute than the reducing agent used for treating the bound genetic display system.

Preferably, the reducing agent in step (a) is preferably included at a concentration of less than 500 μM, preferably less than 200 μM, advantageously less than 100 μM. For example, the reducing agent is present at a concentration of 10 μM or less, such as 1 μM.

The resin-bound polypeptides can be exposed to the reducing agent in purified form, or can be present in culture. Genetic display systems involve replication in cells, such as bacteria or yeast;

these cells can be removed by purification, in which case after the combination of the genetic display system with the purification resin, the polypeptides bound to resin can be washed in buffer and separated from the cell culture contaminants.

Suitably, the genetic display system is eluted from the purification resin after step (b). The polypeptides can then be displayed on the genetic display system in conjugated form, and selected by known means.

The reduction and conjugation/cyclisation reactions are preferably conducted at room temperature, such as 25° C. In some embodiments, the conjugation/cyclisation reaction can be conducted at 30° C. In the aforementioned method of Heinis et al., reactions are conducted at temperatures above room temperature, for example 42° C.

The reduction and conjugation/cyclisation reactions are advantageously conducted for a period of time of less than one hour. For example, the reactions may be conducted for 30 minutes, 20 minutes, 15 minutes or 10 minutes.

In one embodiment, the polypeptide comprises three or more peptide reactive groups covalently linked to a molecular scaffold. Three is the preferred number of peptide reactive groups; four or five groups can also be contemplated. In general, polypeptides with greater number of reactive groups are complex and less amenable to consistent assembly without the formation of isomeric forms.

In one embodiment, the polypeptide is preferably a polypeptide which comprises at least three peptide reactive groups, separated by at least two sequences which can form the “loops” of the polypeptide once conjugated to the molecular scaffold. The loops may be any suitable length, such as two, three, four, five, six, seven or more amino acids long. The loops may be the same length, or different. Preferably, at least two loops are provided. In some embodiments, three, four, five, six or more loops may be present.

The molecular scaffold may be any structure which provides multiple attachment points for the reactive groups of the polypeptide. Exemplary molecular scaffolds are described below. Molecular scaffolds are conjugated to the polypeptide whilst the polypeptides are incorporated into the genetic display system, such that the genetic display system displays the peptide ligand including the molecular scaffold. Excess scaffold is removed.

In certain embodiments, the molecular scaffold is selected from the group of 1,3,5-Tris(bromomethyl)benzene (TBMB), 1,3,5-triacryloyl-1,3,5-triazinane (TATA), 1,1′,1″-(1,4,7-triazonane-1,4,7-triyl)tris(2-chloroethan-1-one) (TCAZ) and 1,1′,1″41H,4H-3a,6a-(methanoiminomethano)pyrrolo[3,4-c]pyrrole-2,5,8(3H,6H)-triyl)tris(2-chloroethan-1-one) (TCCU).

The peptide ligands may be monospecific, binding to a single target molecule, or multispecific. Multispecific peptide ligands are described in WO 2010/089115. The library of peptide ligands may be screened for cross-reactivity between targets from two different species or of two different isotypes.

In embodiments, the peptide ligands are multispecific. In a first configuration, for example, the polypeptide loops formed by the interaction of the polypeptide with the molecular scaffold are capable of binding to more than one target. Within this configuration, in one embodiment loops may be selected individually for binding to the desired targets, and then combined. In another embodiment, the loops are selected together, as part of a single structure, for binding to different desired targets.

In a second configuration, a functional group may be attached to the N or C terminus, or both, of the polypeptide. The functional group may take the form of a binding group, such as a polypeptide, including an antibody domain, an Fc domain or a further structured peptide as described above, capable of binding to a target. It may moreover take the form of a reactive group, capable of chemical bonding with a target. Moreover, it can be an effector group, including large plasma proteins, such as serum albumin, and a cell penetrating peptide.

In a third configuration, a functional group may be attached to the molecular scaffold itself. Examples of functional groups are as for the preceding configuration.

In further embodiments, the peptide ligand comprises a polypeptide linked to a molecular scaffold at n attachment points, wherein said polypeptide is cyclised and forms n separate loops subtended between said n attachment points on the molecular scaffold, wherein n is greater than or equal to 2.

The polypeptide is preferably cyclised by N- to C-terminal fusion, and can be cyclised before or after attachment to the molecular scaffold. Attachment before cyclisation is preferred.

In certain embodiments, the peptide ligand includes at least one loop which comprises a sequence of amino acids subtended between two of the two or more amino acid residues.

Several methods are known in the art for peptide cyclisation. For example, the polypeptide is cyclised by N—C crosslinking, using a crosslinking agent such as EDC.

In another embodiment, the polypeptide can be designed to comprise a protected Na or Ca derivatised amino acid, and cyclised by deprotection of the protected Na or Ca derivatised amino acid to couple said amino acid to the opposite terminus of the polypeptide.

In a preferred embodiment, the polypeptide is cyclised by enzymatic means.

For example, the enzyme is a transglutaminase, for instance a microbial transglutaminase, such as Streptomyces mobaraensis transglutaminase. In order to take advantage of enzymatic cyclisation, it may be necessary to incorporate an N- and/or C-terminal substrate sequence for the enzyme in the polypeptide. Some or all of the substrate sequence(s) can be eliminated during the enzymatic reaction, meaning that the cyclised polypeptide may not comprise the substrate sequences in its final configuration.

### BRIEF DESCRIPTION OF THE FIGURES

FIG. 1: Modification of a phage particle displaying polypeptides with a molecular scaffold to form a peptide ligand. (A) Diagram showing the modification process. (B) Molecular mass of the GCGSGCGSGCG-D1-D2 fusion protein before and after reaction with 10 mM TBMB in 20 mM NH4HCO3, 5 mM EDTA, pH 8, 20% ACN at 30° C. for 1 hour determined by mass spectrometry. The mass difference of the reacted and non-reacted peptide fusion protein corresponds to the mass of the small molecule core mesitylene.

FIG. 2: Peptide-reactive probe assay of the 17-88 single clone phage modified with TBMB using different probe concentrations. One sample each of unmodified and iodoacetamide-capped phage was assayed alongside any scaffolded samples as positive and negative controls respectively.

FIG. 3: Peptide-reactive probe assay of the (A) 3×3; (B) 3×9; (C) 2×7; and (D) 7×2 phage libraries modified with TBMB or TATA using different probe concentrations. One sample each of unmodified and iodoacetamide-capped phage was assayed alongside any scaffolded samples as positive and negative controls respectively.

FIG. 4: Qualification of cyclisation using the peptide-reactive probe assay. (A) Peptide-reactive probe assay of the 6×6 phage library with different ratios of unmodified:TBMB-cyclised phage. (B) Peptide-reactive probe assay of the 6×6 phage library with different ratios of unmodified:TATA-cyclised phage. One sample each of unmodified and iodoacetamide-capped phage was assayed alongside any scaffolded samples as positive and negative controls respectively.

FIG. 5: Peptide-reactive probe assay of (A) the 17-88 single clone modified with TBMB; (B) the 55-28-00 single clone modified with TATA; (C) the 06-663-00 single clone modified with TCAZ; and (D) the 17-69-07 single clone modified with TCCU using different scaffold concentrations. One sample each of unmodified and iodoacetamide-capped phage was assayed alongside any scaffolded samples as positive and negative controls respectively.

FIG. 6: Peptide-reactive probe assay of phage libraries (6×6, 3×3, 3×9, 2×7, 7×2) using the optimised scaffold concentrations (60 μM TBMB, 400 μM TATA, 400 μM TCAZ, 400 μM TCCU). One sample of unmodified phage was assayed alongside as the positive control. The Meleimide-PEG2-Biotin probe concentration used was 100 nM.

FIG. 7: (A) Scaffold-reactive probe assay of single clones (17-88, 541, 542) in which the probe-bound phage was treated with TCEP at different concentrations. One sample of unmodified phage was assayed alongside as the negative control. The SH-PEG3-Biotin probe concentration used was 320 μM. (B) Scaffold-reactive probe assay of unmodified 17-88 single clone phage using different probe concentrations in which the probe-bound phage was treated or not treated with 1 mM TCEP.

FIG. 8: Scaffold-reactive probe assay of (A) the 542 single clone modified with TBMB, TATA, TCAZ or TCCU; (B) the 17-88-PCA5 single clone modified with TBMB or TATA; (C) the FdDog single clone (negative control) modified with TBMB or TATA; (D) the 17-88 single clone modified with TBMB or TATA using different probe concentrations, in which the probe-bound phage was treated with 1 mM TCEP. One sample of unmodified phage was assayed alongside as the negative control.

FIG. 9: Scaffold-reactive probe assay of different single clones (17-88, FdDog, 17-88-PCA3, 17-88-PCA5, 17-88-PCA7) modified with TBMB using different probe concentrations, in which the probe-bound phage was treated with 1 mM TCEP.

FIG. 10: (A) Scaffold-reactive probe assay of the 55-28-02 phage modified with TATA of different concentrations, in which the probe-bound phage was treated with 1 mM TCEP. One sample of unmodified phage was assayed alongside as the negative control. The SH-PEG3-Biotin probe concentration used was 320 μM. (B) Peptide-reactive probe assay of the 55-28-00 phage modified with TATA of different concentrations. One sample of unmodified phage was assayed alongside as the positive control. The Maleimide-PEG2-Biotin probe concentration used was 2.5 μM.

FIG. 11: Scaffold-reactive probe assay of single clones modified with TCAZ, in which the probe-bound phage was treated with 1 mM TCEP. The SH-PEG3-Biotin probe concentration used was 320 μM.

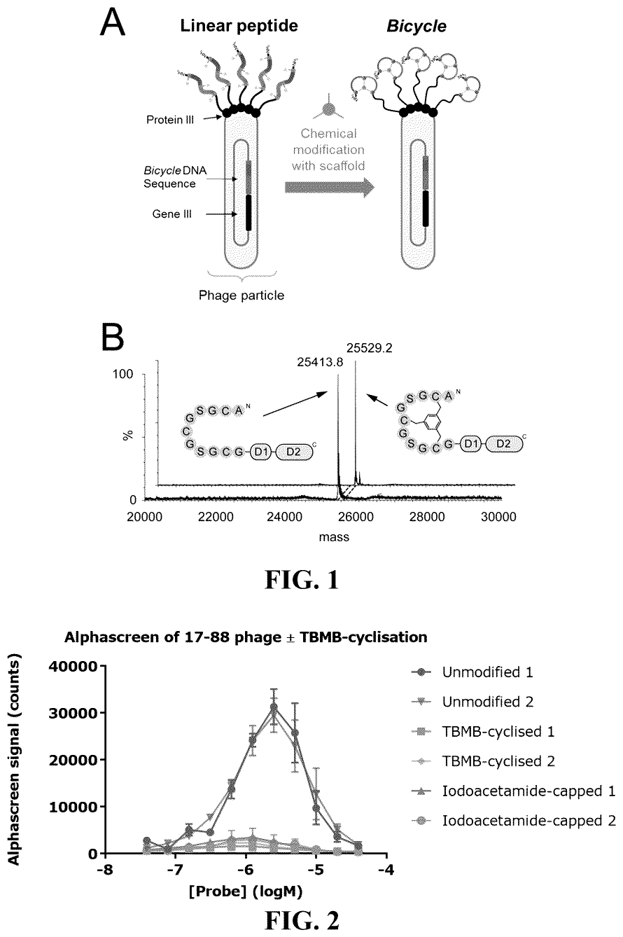

### DETAILED DESCRIPTION OF THE INVENTION

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4ₜₕed., John Wiley & Sons, Inc.), which are incorporated herein by reference.

The “extent of cyclisation” of a peptide ligand refers to the proportion of peptide ligand in which all the two or more peptide reactive groups of a single polypeptide are covalently bound to the two or more scaffold reactive groups of a single molecular scaffold. In general, the peptide ligand is not considered as fully cyclised if:
(1) not all the two or more peptide reactive groups of the polypeptide are conjugated;
(2) the two or more peptide reactive groups of the polypeptide are conjugated to more than one molecular scaffolds;
(3) not all the two or more scaffold reactive groups of the molecular scaffold are conjugated; and/or
(4) the two or more scaffold reactive groups of the molecular scaffold are conjugated to more than one polypeptides.

A (poly)peptide ligand or (poly)peptide conjugate, as referred to herein, refers to a polypeptide covalently bound to a molecular scaffold. Typically, such polypeptides comprise two or more peptide reactive groups which are capable of forming covalent bonds to the molecular scaffold, and a sequence subtended between said reactive groups which is referred to as the loop sequence, since it forms a loop when the polypeptide is bound to the molecular scaffold.

The peptide reactive groups are groups capable of forming a covalent bond with the molecular scaffold. Typically, the peptide reactive groups are present on amino acid side chains on the peptide. Examples are amino-containing groups such as cysteine, lysine, selenocysteine, serine, L-2,3-diaminopropionic acid and N-beta-alkyl-L-2,3-diaminopropionic acid.

The term “probe” can refer to a small molecule, a macromolecule, a polymer, a protein, an antibody or any matter which binds specifically to a target or a target class (e.g. thiol-specific, alkylating agent specific). In the specification, the wording “the probe” can refer to any probes discussed in the present invention.

The term “bind” can refer to binding through covalent bonding, hydrophilic interactions, hydrophobic interactions, van der Waals dispersion forces, dipole-dipole interactions and/or hydrogen bonding.

The term “unconjugated reactive group” can refer to:
(1) the peptide reactive group(s) and/or the scaffold reactive group(s) on the peptide ligand that are not conjugated to the corresponding polypeptide(s) and/or molecular scaffold(s) used in the reaction;
(2) the peptide reactive group(s) on the polypeptide(s) that are not conjugated to the molecular scaffold(s) used in the reaction; and/or
(3) the scaffold reactive group(s) on the molecular scaffold(s) that are not conjugated to the polypeptide(s) used in the reaction.

In the present specification, the term “the unconjugated reactive group” can refer to any unconjugated reactive groups discussed in the present invention.

The term “linkable” can refer to any kind of linkage between the probe and the signalling group/fluorescent entity, such as covalent bonding, hydrophilic interactions, hydrophobic interactions, van der Waals dispersion forces, dipole-dipole interactions and/or hydrogen bonding. In one embodiment, the probe comprises a biotin group and the signalling group/fluorescent entity comprises a streptavidin group.

The term “directly” refers to the situation in which the signal is produced by the signalling group itself. This includes any excitation (such as light and chemicals) that is required to activate, induce or generate such signal.

The term “indirectly” refers to the situation in which the signal is produced by another entity with the assistance of the signalling group. This includes any excitation (such as light and chemicals) that is required to activate or induce such assistance. An entity can be a small molecule, a macromolecule, a polymer or a protein. For example, the signalling group can comprise an enzyme or a catalyst which can catalyse a reaction of a reagent to generate a signal.

The term “indicate” can refer to the determination of the presence of the unconjugated reactive group on the peptide ligand directly or indirectly. Preferably, the signal allows the determination or estimation of the amount, quantity and/or proportion of the unconjugated reactive group on the peptide ligand directly or indirectly.

A fluorescent entity or fluorescent group can refer to a small molecule, a macromolecule, a polymer, a protein or any matter which is fluorescent. In one embodiment, the fluorescent entity or fluorescent group is a fluorescent bead.

Screening for binding or inhibiting activity (or any other desired property) is conducted according to methods well known in the art, for instance from phage display technology. For example, targets immobilised to a solid phase can be used to identify and isolate binding members of a repertoire. Screening allows selection of members of a repertoire according to desired characteristics.

The term library refers to a mixture of heterogeneous polypeptides or nucleic acids. The library is composed of members, which are not identical. To this extent, library is synonymous with repertoire. Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. Preferably, each individual organism or cell contains only one or a limited number of library members.

In one embodiment, the nucleic acids are incorporated into expression vectors, in order to allow expression of the polypeptides encoded by the nucleic acids. In a preferred aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

In one embodiment, a library of nucleic acids encodes a repertoire of polypeptides. Each nucleic acid member of the library preferably has a sequence related to one or more other members of the library. By related sequence is meant an amino acid sequence having at least 50% identity, for example at least 60% identity, for example at least 70% identity, for example at least 80% identity, for example at least 90% identity, for example at least 95% identity, for example at least 98% identity, for example at least 99% identity to at least one other member of the library. Identity can be judged across a contiguous segment of at least 3 amino acids, for example at least 4, 5, 6, 7, 8, 9 or 10 amino acids, for example least 12 amino acids, for example least 14 amino acids, for example least 16 amino acids, for example least 17 amino acids or the full length of the reference sequence.

A repertoire is a collection of variants, in this case polypeptide variants, which differ in their sequence. Typically, the location and nature of the reactive groups will not vary, but the sequences forming the loops between them can be randomised. Repertoires differ in size, but should be considered to comprise at least 10² members. Repertoires of 10¹¹ or more members can be constructed.

Specificity, in the context herein, refers to the ability of a ligand to bind, inhibit or otherwise interact with its cognate target to the exclusion of entities which are similar to the target. For example, specificity can refer to the ability of a ligand to inhibit the interaction of a human enzyme, but not a homologous enzyme from a different species. Using the approach described herein, specificity can be modulated, that is increased or decreased, so as to make the ligands more or less able to interact with homologues or paralogues of the intended target. Specificity is not intended to be synonymous with activity, affinity or avidity, and the potency of the action of a ligand on its target (such as, for example, binding affinity or level of inhibition) are not necessarily related to its specificity.

Binding activity, as used herein, refers to quantitative binding measurements taken from binding assays, for example as described herein. Therefore, binding activity refers to the amount of peptide ligand which is bound at a given target concentration.

Multispecificity is the ability to bind to two or more targets. Typically, binding peptides are capable of binding to a single target, such as an epitope in the case of an antibody, due to their conformational properties. However, peptides can be developed which can bind to two or more targets; dual specific antibodies, for example. In the present invention, the peptide ligands can be capable of binding to two or more target antigens and are therefore be multispecific. Preferably, they bind to two target antigens, and are dual specific. The binding may be independent, which would mean that the binding sites for the targets on the peptide are not structurally hindered by the binding of one or other of the targets. In this case both targets can be bound independently. More generally it is expected that the binding of one target will at least partially impede the binding of the other.

Inhibition, as used herein, refers to the ability of a ligand to bind or interact with a target or a target antigen to reduce its activity or to interfere with its normal function. In the case that the target antigen is an enzyme, the ligand may inhibit by preventing a substrate from entering the enzyme's active site and/or by stopping the enzyme from catalysing a reaction. The ligand may also block the target from interacting with other molecules which are necessary for the normal function of the target. Inhibitory activities (IC₅₀) may be determined by measuring residual activities of the target upon incubation with different concentrations of ligands. Apparent K 1 values may be calculated according to the Cheng and Prusoff equation (Cheng, Y. and Prusoff, W. H., Biochem. Pharmacol., 1973).

A target, an antigen or a target antigen is a molecule or part thereof to which the peptide ligands bind or otherwise interact with. Although binding is seen as a prerequisite to activity of most kinds, and may be an activity in itself, other activities are envisaged. The present invention may not require the measurement of binding directly or indirectly.

(A) Peptide Ligands

(i) Molecular Scaffold

Molecular scaffolds are described in, for example, WO 2009/098450 and references cited therein, particularly WO 2004/077062, WO 2006/078161 and WO 2018/197893.

A molecular scaffold, a molecular core or a scaffold is any molecule which is able to connect the peptide at multiple points to impart one or more structural features to the peptide. It is not a cross-linker, in that it does not merely replace a disulphide bond; instead, it provides two or more attachment points for the peptide. Preferably, the molecular scaffold comprises at least three attachment points for the peptide, referred to as scaffold reactive groups. These groups are capable of reacting to the reactive groups on the peptide to form a covalent bond. Preferably, these groups are capable of reacting with the cysteine residues (Cᵢ, Cᵢᵢ and Cᵢᵢᵢ) on the peptide to form a covalent bond. They do not merely form a disulphide bond, which is subject to reductive cleavage and concomitant disintegration of the molecule, but form stable, covalent thioether linkages. Preferred structures for molecular scaffolds are described below.

The compounds of the invention thus comprise, consist essentially of, or consist of, the peptide covalently bound to a molecular scaffold. The term “scaffold” or “molecular scaffold” herein refers to a chemical moiety that is bonded to the peptide at the alkylamino linkages and thioether linkage in the compounds of the invention. The term “scaffold molecule” or “molecular scaffold molecule” herein refers to a molecule that is capable of being reacted with a peptide or peptide ligand to form the derivatives of the invention having alkylamino and thioether bonds. Thus, the scaffold molecule has the same structure as the scaffold moiety except that respective reactive groups (such as leaving groups) of the molecule are replaced by alkylamino and thioether bonds to the peptide in the scaffold moiety.

The molecular scaffold molecule is any molecule which is able to connect the peptide at multiple points to form the thioether and alkylamino bonds to the peptide. It is not a cross-linker, in that it does not normally link two peptides; instead, it provides two or more attachment points for a single peptide. The molecular scaffold molecule comprises at least three attachment points for the peptide, referred to as scaffold reactive groups. These groups are capable of reacting with —SH and amino groups on the peptide to form the thioether and alkylamino linkages. Thus, the molecular scaffold represents the scaffold moiety up to but not including the thioether and alkylamino linkages in the conjugates of the invention. The scaffold molecule has the structure of the scaffold, but with reactive groups at the locations of the thioether and alkylamino bonds in the conjugate of the invention.

Suitably, the scaffold comprises, consists essentially of, or consists of a (hetero)aromatic or (hetero)alicyclic moiety.

As used herein, “(hetero)aryl” is meant to include aromatic rings, for example, aromatic rings having from 4 to 12 members, such as phenyl rings. These aromatic rings can optionally contain one or more heteroatoms (e.g., one or more of N, O, S, and P), such as thienyl rings, pyridyl rings, and furanyl rings. The aromatic rings can be optionally substituted. “(hetero)aryl” is also meant to include aromatic rings to which are fused one or more other aryl rings or non-aryl rings. For example, naphthyl groups, indole groups, thienothienyl groups, dithienothienyl, and 5,6,7,8-tetrahydro-2-naphthyl groups (each of which can be optionally substituted) are aryl groups for the purposes of the present application. As indicated above, the aryl rings can be optionally substituted. Suitable substituents include alkyl groups (which can optionally be substituted), other aryl groups (which may themselves be substituted), heterocyclic rings (saturated or unsaturated), alkoxy groups (which is meant to include aryloxy groups (e.g., phenoxy groups)), hydroxy groups, aldehyde groups, nitro groups, amine groups (e.g., unsubstituted, or mono- or di-substituted with aryl or alkyl groups), carboxylic acid groups, carboxylic acid derivatives (e.g., carboxylic acid esters, amides, etc.), halogen atoms (e.g., Cl, Br, and I), and the like.

As used herein, “(hetero)alicyclic” refers to a homocyclic or heterocyclic saturated ring. The ring can be unsubstituted, or it can be substituted with one or more substituents. The substituents can be saturated or unsaturated, aromatic or nonaromatic, and examples of suitable substituents include those recited above in the discussion relating to substituents on alkyl and aryl groups. Furthermore, two or more ring substituents can combine to form another ring, so that “ring”, as used herein, is meant to include fused ring systems.

Suitably, the scaffold comprises a tris-substituted (hetero)aromatic or (hetero)alicyclic moiety, for example a tris-methylene substituted (hetero)aromatic or (hetero)alicyclic moiety. The (hetero)aromatic or (hetero)alicyclic moiety is suitably a six-membered ring structure, preferably tris-substituted such that the scaffold has a 3-fold symmetry axis.

In embodiments, the scaffold is a tris-methylene (hetero)aryl moiety, for example a 1,3,5-tris methylene benzene moiety. In these embodiments, the corresponding scaffold molecule suitably has a leaving group on the methylene carbons. The methylene group then forms the R₁ moiety of the alkylamino linkage as defined herein. In these methylene-substituted (hetero)aromatic compounds, the electrons of the aromatic ring can stabilize the transition state during nucleophilic substitution. Thus, for example, benzyl halides are 100-1000 times more reactive towards nucleophilic substitution than alkyl halides that are not connected to a (hetero)aromatic group.

In these embodiments the scaffold and scaffold molecule have the general formula:

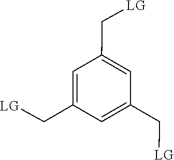

Where LG represents a leaving group as described further below for the scaffold molecule, or LG (including the adjacent methylene group forming the R₁ moiety of the alkylamino group) represents the alkylamino linkage to the peptide in the conjugates of the invention.

In embodiments, the group LG above may be a halogen such as, but not limited to, a bromine atom, in which case the scaffold molecule is 1,3,5-Tris(bromomethyl)benzene (TBMB). Another suitable molecular scaffold molecule is 2,4,6-tris(bromomethyl) mesitylene. It is similar to 1,3,5-tris(bromomethyl) benzene but contains additionally three methyl groups attached to the benzene ring. In the case of this scaffold, the additional methyl groups may form further contacts with the peptide and hence add additional structural constraint. Thus, a different diversity range is achieved than with 1,3,5-Tris(bromomethyl)benzene.

Another preferred molecule for forming the scaffold for reaction with the peptide by nucleophilic substitution is 1,3,5-tris(bromoacetamido)benzene (TBAB):

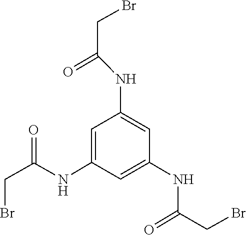 [SMILES: O=C(CBr)Nc1cc(NC(=O)CBr)cc(NC(=O)CBr)c1]

In embodiments, the scaffold molecule comprises a (hetero)alicyclic moiety, preferably a tris-substituted (hetero)alicyclic moiety, more preferably a tris-(2-haloethan-1-one) (hetero)alicyclic moiety.

A preferred molecule for forming the scaffold for reaction with the peptide by nucleophilic substitution is 1,1′,1″-(1,4,7-triazonane-1,4,7-triyl)tris(2-chloroethan-1-one) (TCAZ):

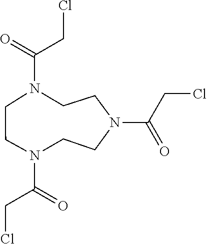 [SMILES: O=C(CCl)N1CCN(C(=O)CCl)CCN(C(=O)CCl)CC1]

Another preferred molecule for forming the scaffold for reaction with the peptide by nucleophilic substitution is 1,1′,1″-[1H,4H-3a,6a-(methanoiminomethano)pyrrolo[3,4-c]pyrrole-2,5,8(3H,6H)-triyl]tris(2-chloroethan-1-one) (TCCU):

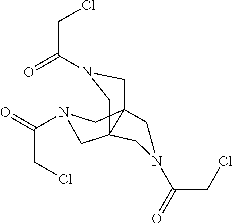 [SMILES: O=C(CCl)N1CC23CN(C(=O)CCl)CC2(C1)CN(C(=O)CCl)C3]

In other embodiments the molecular scaffold may have a tetrahedral geometry such that reaction of four functional groups of the encoded peptide with the molecular scaffold generates not more than two product isomers. Other geometries are also possible; indeed, an almost infinite number of scaffold geometries is possible, leading to greater possibilities for peptide ligand diversification.

In other embodiments, the scaffold molecule may be a (hetero)aromatic or (hetero)alicyclic moiety substituted with two or more acryloyl groups, such as acrylamide or acrylate groups. These groups can undergo α,β-addition reactions with —SH to form thioether bonds. A typical scaffold molecule of this type is 1,3,5-triacryloyl-1,3,5-triazinane (TATA):

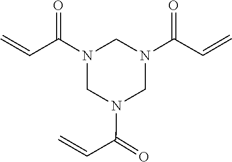 [SMILES: C=CC(=O)N1CN(C(=O)C=C)CN(C(=O)C=C)C1]

In yet other embodiments the molecular scaffold may have a tetrahedral geometry such that reaction of four functional groups of the encoded peptide with the molecular scaffold generates not more than two product isomers. Other geometries are also possible; indeed, an almost infinite number of scaffold geometries is possible, leading to greater possibilities for peptide derivative diversification.

The peptides used to form the ligands of the invention can comprise Dap or N-AlkDap residues for forming alkylamino linkages to the scaffold. The structure of diaminopropionic acid is analogous to and isosteric that of cysteine that has been used to form thioether bonds to the scaffold in the prior art, with replacement of the terminal —SH group of cysteine by —NH₂:

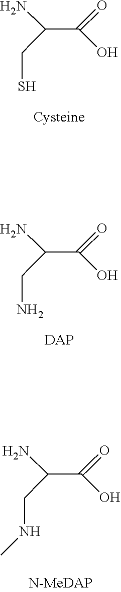

The term “alkylamino” is used herein in its normal chemical sense to denote a linkage consisting of NH or N(R₃) bonded to two carbon atoms, wherein the carbon atoms are independently selected from alkyl, alkylene, or aryl carbon atoms and R₃ is an alkyl group. Suitably, the alkylamino linkages of the invention comprise an NH moiety bonded to two saturated carbon atoms, most suitably methylene (—CH₂—) carbon atoms. The alkylamino linkages useful in the invention have general formula:

S—R₁—N(R₃)—R₂—P
Wherein:
S represents the scaffold core, e.g. a (hetero)aromatic or (hetero)alicyclic ring as explained further below;
R₁ is C1 to C3 alkylene groups, suitably methylene or ethylene groups, and most suitably methylene (CH₂);
R₂ is the methylene group of the Dap or N-AlkDap side chain
R₃ is C₁-4 alkyl including branched alkyl and cycloalkyl, for example methyl, or H; and
P represents the peptide backbone, i.e. the R₂ moiety of the above linkage is linked to the carbon atom in the peptide backbone adjacent to a carboxylic carbon of the Dap or N-AlkDap residue.

(ii) Polypeptide

In the present invention, the terms “peptide” and “polypeptide” are used interchangeably.

The peptide reactive groups of the polypeptides can be provided by side chains of natural or non-natural amino acids. The peptide reactive groups of the polypeptides can be selected from thiol groups, amino groups, carboxyl groups, guanidinium groups, phenolic groups or hydroxyl groups. The peptide reactive groups of the polypeptides can be selected from azide, keto-carbonyl, alkyne, vinyl, or aryl halide groups. The peptide reactive groups of the polypeptides for linking to a molecular scaffold can be the amino or carboxy termini of the polypeptide. Corresponding scaffold reactive groups can be used on the molecular scaffold to react with the above peptide reactive groups. Further details can be found in WO 2009/098450.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine.

Cysteine can be employed because it has the advantage that its reactivity is most different from all other amino acids. Scaffold reactive groups that could be used on the molecular scaffold to react with thiol groups of cysteines are alkyl halides (or also named halogenoalkanes or haloalkanes). Examples are bromomethylbenzene (the scaffold reactive group exemplified by TBMB) or iodoacetamide. Other scaffold reactive groups that are used to couple selectively compounds to cysteines in proteins are maleimides. Examples of maleimides which may be used as molecular scaffolds in the invention include: tris-(2-maleimidoethyl)amine, tris-(2-maleimidoethyl)benzene, tris-(maleimido)benzene. Other possible scaffold reactive groups include α-halocarbonyls, vinyl sulfones, alkene (thiol-ene coupling), alkyne (thiol-yne coupling), thiol (disulphide reaction) and other bioconjugating agents known in the art. Selenocysteine is also a natural amino acid which has a similar reactivity to cysteine and can be used for the same reactions. Thus, wherever cysteine is mentioned, it is typically acceptable to substitute selenocysteine unless the context suggests otherwise.

Lysines (and primary amines of the N-terminus of peptides) are also suited as peptide reactive groups to modify peptides on phage by linking to a molecular scaffold. However, they are more abundant in phage proteins than cysteines and there is a higher risk that phage particles might become cross-linked or that they might lose their infectivity. Nevertheless, it has been found that lysines are especially useful in intramolecular reactions (e.g. when a molecular scaffold is already linked to the phage peptide) to form a second or consecutive linkage with the molecular scaffold. In this case the molecular scaffold reacts preferentially with lysines of the displayed peptide (in particular lysines that are in close proximity). Scaffold reactive groups that react selectively with primary amines are succinimides, aldehydes, isocyanate, isothiocyanate, sulfonyl halides, sulfonates, aryl halides, imidoesters, alkyl halides or any other bioconjugating reagents known in the art. In the bromomethyl group that is used in a number of the accompanying examples, the electrons of the benzene ring can stabilize the cationic transition state. This particular aryl halide is therefore 100-1000 times more reactive than alkyl halides. Examples of succinimides for use as molecular scaffold include tris-(succinimidyl aminotriacetate), 1,3,5-Benzenetriacetic acid. Examples of aldehydes for use as molecular scaffold include Triformylmethane. Examples of alkyl halides for use as molecular scaffold include 1,3,5-Tris(bromomethyl)-2,4,6-trimethylbenzene, 1,3,5-Tris(bromomethyl) benzene, 1,3,5-Tris(bromomethyl)-2,4,6-triethylbenzene.

The polypeptides of the invention contain at least two peptide reactive groups. Said polypeptides can also contain three or more peptide reactive groups. Said polypeptides can also contain four or more peptide reactive groups. The more peptide reactive groups are used, the more loops can be formed in the molecular scaffold.

In a preferred embodiment, polypeptides with three peptide reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesised. In this case, the chemical reaction of the polypeptide with the molecular scaffold yields a single product isomer rather than a mixture of isomers.

In another embodiment of the invention, polypeptides with four peptide reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesising both isomers, separating the two isomers and testing both isomers for binding to a target ligand.

In some embodiments each of the peptide reactive groups of the polypeptide for linking to a molecular scaffold are of the same type. For example, each peptide reactive group may be a cysteine residue. Further details are provided in WO 2009/098450.

In some embodiments the peptide reactive groups for linking to a molecular scaffold may comprise two or more different types, or may comprise three or more different types. For example, the peptide reactive groups may comprise two cysteine residues and one lysine residue, or may comprise one cysteine residue, one lysine residue and one N-terminal amine.

In one embodiment of the invention, at least one of the peptide reactive groups of the polypeptides is orthogonal to the remaining reactive groups. The use of orthogonal peptide reactive groups allows the directing of said orthogonal peptide reactive groups to specific sites of the molecular core. Linking strategies involving orthogonal peptide reactive groups may be used to limit the number of product isomers formed. In other words, by choosing distinct or different peptide reactive groups for one or more of the at least three bonds to those chosen for the remainder of the at least three bonds, a particular order of bonding or directing of specific reactive groups of the polypeptide to specific positions on the molecular scaffold may be usefully achieved.

In another embodiment, the peptide reactive groups of the polypeptide of the invention are reacted with molecular linkers wherein said linkers are capable to react with a molecular scaffold so that the linker will intervene between the molecular scaffold and the polypeptide in the final bonded state.

In some embodiments, amino acids of the members of the libraries or sets of polypeptides can be replaced by any natural or non-natural amino acid. Excluded from these exchangeable amino acids are the ones harbouring functional groups for cross-linking the polypeptides to a molecular core, such that the loop sequences alone are exchangeable. The exchangeable polypeptide sequences have either random sequences, constant sequences or sequences with random and constant amino acids. The amino acids with reactive groups are either located in defined positions within the polypeptide, since the position of these amino acids determines loop size.

The amino acids with peptide reactive groups for linking to a molecular scaffold may be located at any suitable positions within the polypeptide. In order to influence the particular structures or loops created, the positions of the amino acids having the peptide reactive groups can be varied by the skilled operator, e.g. by manipulation of the nucleic acid encoding the polypeptide in order to mutate the polypeptide produced. By such means, loop length can be manipulated in accordance with the present teaching.

For example, the polypeptide can comprise the sequence AC(X)ₙC(X)ₘCG, wherein X stands for a random amino acid, A for alanine, C for cysteine and G for glycine and n and m, which may be the same or different, are numbers between 2 and 15, and in embodiments may be between 2 and 10, 2 and 9, 2 and 7 or 2 and 6.

In one embodiment, a polypeptide with three peptide reactive groups has the sequence (X)ₗY(X)ₘY(X)ₙY(X)ₒ, wherein Y represents an amino acid with a reactive group, X represents a random amino acid, m and n are numbers between 2 and 9 defining the length of intervening polypeptide segments, which may be the same or different, and 1 and o are numbers between 0 and 20 defining the length of flanking polypeptide segments.

Alternatives to thiol-mediated conjugations can be used to attach the molecular scaffold to the peptide via covalent interactions. Alternatively these techniques can be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods can be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis, et al., Nat Chem Biol 2009, 5 (7), 502-7.

(iii) Combination of Loops to Form Multispecific Molecules

Loops from peptide ligands, or repertoires of peptide ligands, are advantageously combined by sequencing and de novo synthesis of a polypeptide incorporating the combined loops. Alternatively, nucleic acids encoding such polypeptides can be synthesised.

Where repertoires are to be combined, particularly single loop repertoires, the nucleic acids encoding the repertoires are advantageously digested and re-ligated, to form a novel repertoire having different combinations of loops from the constituent repertoires. Phage vectors can include polylinkers and other sites for restriction enzymes which can provide unique points for cutting and relegation the vectors, to create the desired multispecific peptide ligands. Methods for manipulating phage libraries are well known in respect of antibodies, and can be applied in the present case also.

(iv) Attachment of Effector Groups and Functional Groups

Effector and/or functional groups can be attached, for example, to the N or C termini of the polypeptide, or to the molecular scaffold.

Appropriate effector groups include antibodies and parts or fragments thereof. For instance, an effector group can include an antibody light chain constant region (CL), an antibody CHI heavy chain domain, an antibody CH₂ heavy chain domain, an antibody CH₃ heavy chain domain, or any combination thereof, in addition to the one or more constant region domains. An effector group may also comprise a hinge region of an antibody (such a region normally being found between the CHI and CH₂ domains of an IgG molecule).

In an embodiment, an effector group according to the present invention is an Fc region of an IgG molecule. Advantageously, a peptide ligand-effector group according to the present invention comprises or consists of a peptide ligand Fc fusion having a tβ half-life of a day or more, two days or more, 3 days or more, 4 days or more, 5 days or more, 6 days or more or 7 days or more. Most advantageously, the peptide ligand according to the present invention comprises or consists of a peptide ligand Fc fusion having a tβ half-life of a day or more.

Functional groups include, in general, binding groups, drugs, reactive groups for the attachment of other entities, functional groups which aid uptake of the macrocyclic peptides into cells, and the like.

The ability of peptides to penetrate into cells will allow peptides against intracellular targets to be effective. Targets that can be accessed by peptides with the ability to penetrate into cells include transcription factors, intracellular signalling molecules such as tyrosine kinases and molecules involved in the apoptotic pathway. Functional groups which enable the penetration of cells include peptides or chemical groups which have been added either to the peptide or the molecular scaffold. Peptides such as those derived from such as VP22, HIV-Tat, a homeobox protein of Drosophila (Antennapedia), e.g. as described in Chen and Harrison, Biochemical Society Transactions (2007) Volume 35, part 4, p821 “Cell-penetrating peptides in drug development: enabling intracellular targets” and “Intracellular delivery of large molecules and small peptides by cell penetrating peptides” by Gupta et al. in Advanced Drug Discovery Reviews (2004) Volume 57 9637. Examples of short peptides which have been shown to be efficient at translocation through plasma membranes include the 16 amino acid penetratin peptide from Drosophila Antennapedia protein (Derossi, et al. (1994) J Biol. Chem. Volume 269 p10444 “The third helix of the Antennapedia homeodomain translocates through biological membranes”), the 18 amino acid ‘model amphipathic peptide’ (Oehlke, et al. (1998) Biochim Biophys Acts Volume 1414 p127 “Cellular uptake of an alpha-helical amphipathic model peptide with the potential to deliver polar compounds into the cell interior non-endocytically”) and arginine rich regions of the HIV TAT protein. Non peptidic approaches include the use of small molecule mimics or SMOCs that can be easily attached to biomolecules (Okuyama, et al. (2007) Nature Methods Volume 4 p153 ‘Small-molecule mimics of an α-helix for efficient transport of proteins into cells’. Other chemical strategies to add guanidinium groups to molecules also enhance cell penetration (Elson-Scwab, et al. (2007) J Biol Chem Volume 282 p13585 “Guanidinylated Neomcyin Delivers Large Bioactive Cargo into cells through a heparin Sulphate Dependent Pathway”). Small molecular weight molecules such as steroids may be added to the molecular scaffold to enhance uptake into cells.

One class of functional groups which may be attached to peptide ligands includes antibodies and binding fragments thereof, such as Fab, Fv or single domain fragments. In particular, antibodies which bind to proteins capable of increasing the half-life of the peptide ligand in vivo may be used. RGD peptides, which bind to integrins which are present on many cells, may also be incorporated.

In one embodiment, a peptide ligand-effector group according to the invention has a tβ half-life selected from the group consisting of: 12 hours or more, 24 hours or more, 2 days or more, 3 days or more, 4 days or more, 5 days or more, 6 days or more, 7 days or more, 8 days or more, 9 days or more, 10 days or more, 11 days or more, 12 days or more, 13 days or more, 14 days or more, days or more or 20 days or more. Advantageously a peptide ligand-effector group or composition according to the invention will have a tβ half life in the range 12 to 60 hours. In a further embodiment, it will have a t half-life of a day or more. In a further embodiment still, it will be in the range 12 to 26 hours.

Functional groups include drugs, such as cytotoxic agents for cancer therapy. These include alkylating agents such as Cisplatin and carboplatin, as well as oxaliplatin, mechlorethamine, cyclophosphamide, chlorambucil, ifosfamide; Anti-metabolites including purine analogs azathioprine and mercaptopurine or pyrimidine analogs; plant alkaloids and terpenoids including vinca alkaloids such as Vincristine, Vinblastine, Vinorelbine and Vindesine; Podophyllotoxin and its derivatives etoposide and teniposide; Taxanes, including paclitaxel, originally known as Taxol; topoisomerase inhibitors including camptothecins: irinotecan and topotecan, and type II inhibitors including amsacrine, etoposide, etoposide phosphate, and teniposide. Further agents can include Antitumour antibiotics which include the immunosuppressant dactinomycin (which is used in kidney transplantations), doxorubicin, epirubicin, bleomycin and others.

Possible effector groups also include enzymes, for instance such as carboxypeptidase G2 for use in enzyme/prodrug therapy, where the peptide ligand replaces antibodies in ADEPT.

(v) Peptide Modification

To develop the peptide ligands or the bicyclic peptides (Bicycles; peptides conjugated to molecular scaffolds) into a suitable drug-like molecule, whether that is for injection, inhalation, nasal, ocular, oral or topical administration, a number of properties need to be considered. The following at least need to be designed into a given lead Bicycle:
protease stability, whether this concerns Bicycle stability to plasma proteases, epithelial (“membrane-anchored”) proteases, gastric and intestinal proteases, lung surface proteases, intracellular proteases and the like. Protease stability should be maintained between different species such that a Bicycle lead candidate can be developed in animal models as well as administered with confidence to humans.
replacement of oxidation-sensitive residues, such as tryptophan and methionine with oxidation-resistant analogues in order to improve the pharmaceutical stability profile of the molecule
a desirable solubility profile, which is a function of the proportion of charged and hydrophilic versus hydrophobic residues, which is important for formulation and absorption purposes
correct balance of charged versus hydrophobic residues, as hydrophobic residues influence the degree of plasma protein binding and thus the concentration of the free available fraction in plasma, while charged residues (in particular arginines) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic residues may reduce irritation at the injection site (were the peptide drug administered subcutaneously).
a tailored half-life, depending on the clinical indication and treatment regimen. It may be prudent to develop an unmodified molecule for short exposure in an acute illness management setting, or develop a bicyclic peptide with chemical modifications that enhance the plasma half-life, and hence be optimal for the management of more chronic disease states.

Approaches to stabilise therapeutic peptide candidates against proteolytic degradation are numerous, and overlap with the peptidomimetics field (for reviews see Gentilucci et al, Curr. Pharmaceutical Design, (2010), 16, 3185-203, and Nestor et al, Curr. Medicinal Chem (2009), 16, 4399-418).

These include:
Cyclisation of peptide
N- and C-terminal capping, usually N-terminal acetylation and C-terminal amidation.
Alanine scans, to reveal and potentially remove the proteolytic attack site(s).
D-amino acid replacement, to probe the steric requirements of the amino acid side chain, to increase proteolytic stability by steric hindrance and by a propensity of D-amino acids to stabilise β-turn conformations (Tugyi, et al. (2005) PNAS, 102(2), 413-418).
N-methyl/N-alkyl amino acid replacement, to impart proteolytic protection by direct modification of the scissile amide bond (Fiacco, et al., Chembiochem. (2008), 9(14), 2200-3). N-methylation also has strong effect on the torsional angles of the peptide bond, and is believed to aid in cell penetration & oral availability (Biron, et al. (2008), Angew. Chem. Int. Ed., 47, 2595-99)
Incorporation of non-natural amino acids, i.e. by employing
   Isosteric/isoelectronic side chains that are not recognised by proteases, yet have no effect on target potency
   Constrained amino acid side chains, such that proteolytic hydrolysis of the nearby peptide bond is conformationally and sterically impeded. In particular, these concern proline analogues, bulky sidechains, Ca-disubstituted derivatives (where the simplest derivative is Aib, H₂N—C(CH₃)₂—COOH), and cyclo amino acids, a simple derivative being amino-cyclopropylcarboxylic acid).
Peptide bond surrogates, and examples include
   N-alkylation (see above, i.e. CO—NR)
   Reduced peptide bonds (CH₂—NH—)
   Peptoids (N-alkyl amino acids, NR—CH₂—CO)
   Thio-amides (CS—NH)
   Azapeptides (CO—NH—NR)
   Trans-alkene (RHC═C—)
   Retro-inverso (NH—CO)
   Urea surrogates (NH—CO—NHR)
Peptide backbone length modulation
   i.e. β^{2/3}-amino acids, (NH—CR—CH₂—CO, NH—CH₂—CHR—CO),
Substitutions on the alpha-carbon on amino acids, which constrains backbone conformations, the simplest derivative being Aminoisobutyric acid (Aib).

It should be explicitly noted that some of these modifications may also serve to deliberately improve the potency of the peptide against the target, or, for example to identify potent substitutes for the oxidation-sensitive amino acids (Trp and Met).

The invention also relates to peptide ligands having more than two loops. For example, tricyclic polypeptides joined to a molecular scaffold can be created by joining the N- and C-termini of a bicyclic polypeptide joined to a molecular scaffold according to the present invention. In this manner, the joined N and C termini create a third loop, making a tricyclic polypeptide. This embodiment need not be carried out on phage, but can be carried out on a polypeptide-molecular scaffold conjugate as described herein. Joining the N- and C-termini is a matter of routine peptide chemistry. In case any guidance is needed, the C-terminus may be activated and/or the N- and C-termini may be extended for example to add a cysteine to each end and then join them by disulphide bonding. Alternatively the joining may be accomplished by use of a linker region incorporated into the N/C termini. Alternatively the N and C termini may be joined by a conventional peptide bond. Alternatively any other suitable means for joining the N and C termini may be employed, for example N—C-cyclization could be done by standard techniques, for example as disclosed in Linde, et al. Peptide Science 90, 671-682 (2008)-“Structure-activity relationship and metabolic stability studies of backbone cyclization and N-methylation of melanocortin peptides”, or as in Hess, et al. J. Med. Chem. 51, 1026-1034 (2008)-“backbone cyclic peptidomimetic melanocortin-4 receptor agonist as a novel orally administered drug lead for treating obesity”. One advantage of such tricyclic molecules is the avoidance of proteolytic degradation of the free ends, in particular by exoprotease action. Another advantage of a tricyclic polypeptide of this nature is that the third loop may be utilised for generally applicable functions such as BSA binding, cell entry or transportation effects, tagging or any other such use. It will be noted that this third loop will not typically be available for selection (because it is not produced on the phage but only on the polypeptide-molecular scaffold conjugate) and so its use for other such biological functions still advantageously leaves both loops 1 and 2 for selection/creation of specificity.

(B) Repertoires, Sets and Groups of Polypeptide Ligands

(i) Construction of Libraries

Libraries intended for selection may be constructed using techniques known in the art, for example as set forth in WO 2004/077062, or biological systems, including phage vector systems as described herein. Other vector systems are known in the art, and include other phage (for instance, phage lambda), bacterial plasmid expression vectors, eukaryotic cell-based expression vectors, including yeast vectors, and the like. For example, see WO 2009/098450 or Heinis, et al., Nat Chem Biol 2009, 5 (7), 502-7.

Non-biological systems such as those set forth in WO 2004/077062 are based on conventional chemical screening approaches. They are simple, but lack the power of biological systems since it is impossible, or at least impracticably onerous, to screen large libraries of peptide ligands. However, they are useful where, for instance, only a small number of peptide ligands needs to be screened. Screening by such individual assays, however, may be time-consuming and the number of unique molecules that can be tested for binding to a specific target generally does not exceed 6 chemical entities.

In contrast, biological screening or selection methods generally allow the sampling of a much larger number of different molecules. Thus biological methods can be used in application of the invention. In biological procedures, molecules are assayed in a single reaction vessel and the ones with favourable properties (i.e. binding) are physically separated from inactive molecules. Selection strategies are available that allow to generate and assay simultaneously more than 10¹³ individual compounds. Examples for powerful affinity selection techniques are phage display, ribosome display, mRNA display, yeast display, bacterial display or RNA/DNA aptamer methods. These biological in vitro selection methods have in common that ligand repertoires are encoded by DNA or RNA. They allow the propagation and the identification of selected ligands by sequencing. Phage display technology has for example been used for the isolation of antibodies with very high binding affinities to virtually any target.

When using a biological system, once a vector system is chosen and one or more nucleic acid sequences encoding polypeptides of interest are cloned into the library vector, one may generate diversity within the cloned molecules by undertaking mutagenesis prior to expression; alternatively, the encoded proteins may be expressed and selected before mutagenesis and additional rounds of selection are performed.

Mutagenesis of nucleic acid sequences encoding structurally optimised polypeptides is carried out by standard molecular methods. Of particular use is the polymerase chain reaction, or PCR, (Mullis and Faloona (1987) Methods Enzymol., 155: 335, herein incorporated by reference). PCR, which uses multiple cycles of DNA replication catalysed by a thermostable, DNA-dependent DNA polymerase to amplify the target sequence of interest, is well known in the art. The construction of various antibody libraries has been discussed in Winter, et al. (1994) Ann. Rev. Immunology 12, 433-55, and references cited therein.

Alternatively, given the short chain lengths of the polypeptides according to the invention, the variants are preferably synthesised de novo and inserted into suitable expression vectors. Peptide synthesis can be carried out by standard techniques known in the art, as described above. Automated peptide synthesizers are widely available, such as the Applied Biosystems ABI 433 (Applied Biosystems, Foster City, CA, USA)

(ii) Genetically Encoded Diversity

In one embodiment, the polypeptides of interest are genetically encoded. This offers the advantage of enhanced diversity together with ease of handling. An example of a genetically polypeptide library is a mRNA display library. Another example is a replicable genetic display package (rgdp) library such as a phage display library. In one embodiment, the polypeptides of interest are genetically encoded as a phage display library. Thus, in one embodiment the complex of the invention comprises a replicable genetic display package (rgdp) such as a phage particle. In these embodiments, the nucleic acid can be comprised by the phage genome. In these embodiments, the polypeptide can be comprised by the phage coat.

In some embodiments, the invention may be used to produce a genetically encoded combinatorial library of polypeptides which are generated by translating a number of nucleic acids into corresponding polypeptides and linking molecules of said molecular scaffold to said polypeptides.

The genetically encoded combinatorial library of polypeptides may be generated by phage display, yeast display, ribosome display, bacterial display or mRNA display.

Techniques and methodology for performing phage display can be found in WO 2009/098450.

In one embodiment, screening may be performed by contacting a library, set or group of polypeptide ligands with a target and isolating one or more member(s) that bind to said target.

In another embodiment, individual members of said library, set or group are contacted with a target in a screen and members of said library that bind to said target are identified.

In another embodiment, members of said library, set or group are simultaneously contacted with a target and members that bind to said target are selected.

The target(s) may be a peptide, a protein, a polysaccharide, a lipid, a DNA or a RNA.

The target may be a receptor, a receptor ligand, an enzyme, a hormone or a cytokine.

The target may be a prokaryotic protein, a eukaryotic protein, or an archeal protein. More specifically the target ligand may be a mammalian protein or an insect protein or a bacterial protein or a fungal protein or a viral protein.

The target ligand may be an enzyme, such as a protease.

It should be noted that the invention also embraces polypeptide ligands isolated from a screen according to the invention. In one embodiment the screening method(s) of the invention further comprise the step of: manufacturing a quantity of the polypeptide isolated as capable of binding to said targets.

(iii) Phage Purification

In accordance with the present invention, phage purification before reaction with the molecular scaffold is optional. In the event that purification is desired, any suitable means for purification of the phage may be used. Standard techniques may be applied in the present invention. For example, phage may be purified by filtration or by precipitation such as PEG precipitation; phage particles may be produced and purified by polyethylene-glycol (PEG) precipitation as described previously. Details can be found in WO 2009/098450.

In case further guidance is needed, reference is made to Jespers et al (Protein Engineering Design and Selection 2004 17(10):709-713. Selection of optical biosensors from chemisynthetic antibody libraries.) In one embodiment phage may be purified as taught therein. The text of this publication is specifically incorporated herein by reference for the method of phage purification;

in particular reference is made to the materials and methods section starting part way down the right-column at page 709 of Jespers et al.

Moreover, the phage may be purified as published by Marks et al, J. Mol. Biol vol 222 pp 581-597, which is specifically incorporated herein by reference for the particular description of how the phage production/purification is carried out.

If phage purification is not desired, culture medium including phage can be mixed directly with a purification resin and a reducing agent (such as TCEP), as set forth in the examples herein.

(iv) Reaction Chemistry

The reaction chemistry can be that set forth in WO 2009/098450 by Heinis et al., or, preferably, that set forth in WO 2014/140342. Reactions conditions used in the present invention preferably comprise the following steps, all preferably conducted at room temperature:
1. Culture medium from which bacterial cells have been removed, containing phage expressing the desired polypeptide(s), is mixed with buffer, reducing agent and resin equilibrated in buffer.
2. The resin is isolated and resuspended in buffer and dilute reducing agent.
3. The polypeptides are exposed to the molecular scaffold and reacted therewith such that the molecular scaffold forms covalent bonds with the polypeptide.
4. The samples are washed to remove excess unreacted scaffold.
5. Phage are eluted from the resin.

The buffer is preferably pH 8.0; it is not necessary to adjust buffer pH in the final solution. Suitable buffers include NaHCO₃, initially at pH 8.0. Alternative buffers may be used, including buffers with a pH in the physiological range, including NH₄CO₃, HEPES and Tris-hydroxymethyl aminoethane, Tris, Tris-Acetate or MOPS. The NaHCO₃ buffer is preferably used at a concentration of 1 M, adding 1 ml to a suspension of resin to equilibrate the resin.

The resin is preferably an ion exchange resin. Ion exchange resins are known in the art, and include any material suitable for anion exchange chromatography known in the art, such as an agarose based chromatography material, e.g. sepharoses like Fast Flow or Capto, polymeric synthetic material, e.g. a polymethacrylate such as Toyopearls, polystyrene/divinylbenzene, such as Poros, Source, or cellulose, e.g. Cellufine. In a preferred embodiment, the anion exchange resin material includes, but is not limited to a resin that carries a primary amine as ligand, e.g. aminohexyl sepharose, benzamidine sepharose, lysine sepharose, or arginine sepharose. In another preferred embodiment, the anion exchange resin material includes, but is not limited to a resin having a positively charged moiety at neutral pH, such as alkylaminoethane, like diethylaminoethane (DEAE), dimethylaminoethane (DMAE), or trimethylaminoethyl (TMAE), polyethyleneimine (PEI), quaternary aminoalkyl, quaternary aminoethane (QAE), quaternary ammonium (Q), and the like.

In step (1), reducing agent is added to a concentration of 1 mM. The dilute reducing agent used in step (2) is preferably at a concentration of 1 μM. Both concentrations are for TCEP, and other values may apply to other reducing agents. The dilute reducing agent is used to maintain the polypeptide in a reduced state prior to reaction with the molecular scaffold. Preferably, a chelating agent is included in the washing step. For example, EDTA may be included.

Alternative reducing agents may be selected from dithiothreitol, thioglycolic acid, thiolactic acid, 3-mercaptopropionic acid, thiomalic acid, 2,3-dimercaptosuccinic acid, cysteine, N-glycyi-L-cysteine, L-cysteinylglycine and also esters and salts thereof, thioglycerol, cysteamine and C1-C4 acyl derivatives thereof, N-mesylcysteamine, Nacetylcysteine, N-mercaptoalkylamides of sugars such as N-(mercapto-2-ethyl) gluconamide, pantetheine, N-(mercaptoalkyl)-co-hydroxyalkylamides, for example those described in patent application EP-A-354 835, N-mono- or N,N-dialkylmercapto-4-butyramides, for example those described in patent application EP-A-368 763, aminomercaptoalkyl amides, for example those described in patent application EP-A-432 000, N-(mercaptoalkyl)succinamic acids and N-(mercaptoalkyl)succinimides, for example those described in patent application EP-A-465 342, alkylamine mercaptoalkyl amides, for example those described in patent application EP-A-514 282, the azeotropic mixture of 2-hydroxypropyl thioglycolate and of (2-hydroxy-1-methyl)ethyl thioglycolate as described in patent application FR-A-2 679 448, mercaptoalkylamino amides, for example those described in patent application FR-A-2 692 481, and N-mercaptoalkylalkanediamides, for example those described in patent application EP-A-653 202.

The conjugation of the molecular scaffold, in the case of TBMB and other scaffolds whose reactive groups are thiol-reactive, is preferably conducted in the presence of acetonitrile. The acetonitrile is preferably at a final concentration of about 20%.

Alternative scaffolds to TBMB are discussed herein.

Unreacted molecular scaffold is removed from the phage by washing. Subsequently, phage can be eluted from the resin, and selected as set forth previously.

Additional steps can also be included in the procedure. Such steps are not mandatory, and do not significantly increase the yield or efficiency of the process.

For example, the phage-containing culture medium, combined with the resin, can be washed prior to reduction with the reducing agent. The reducing agent itself can be added in two steps; in a concentrated form, to effect reduction, and then in dilute form (step 2 above), to maintain the displayed polypeptide in a reduced state.

The timing of the steps can also be varied, without significantly altering the efficiency of the procedure. For example, it has been found that reduction in TCEP for 20 minutes is as effective as reduction for 30 minutes. Likewise, reaction with TBMB for 10 minutes does not give a significantly lower level of binding than reaction for 30 minutes.

(v) Magnetic Separation

In an advantageous embodiment, the resin is magnetic. This allows the polypeptide-bearing phage to be isolated by magnetic separation. Magnetic resin beads, such as magnetic sepharose beads, can be obtained commercially from, for example, Bangs Laboratories, Invitrogen, Origene and GE Healthcare. See also U.S. Pat. No. 2,642,514 and GB 1239978. Application of a magnetic field permits isolation of the beads, which results in purification of the polypeptides bound to the beads from the medium in which they are contained.

In one embodiment, the magnetic beads are separated from the medium by insertion of a magnetic probe into the medium. Beads are retained on the magnetic probe, and can be transferred to a washing station, or a different medium. Alternatively, beads can be isolated by applying a magnetic field to the vessel in which they are contained, and removing the medium once the beads are immobilised.

Magnetic separation provides faster, more efficient processing of resins in the method of the invention.

(C) Probes

(i) Probe Reactive Groups

In the present invention, a probe generally comprises a probe reactive group which binds to one of the following:
(1) one of the two or more peptide reactive groups on the peptide ligand;
(2) one of the two or more scaffold reactive groups on the peptide ligand; and
(3) the genetic display system.

With regard to (1) and (2), the probe reactive group can be similar or identical to the scaffold reactive group and the peptide reactive group respectively.

Probe reactive groups that could be used to react with thiol groups of cysteines include but are not limited to alkyl halides (or also named halogenoalkanes or haloalkanes), maleimides, α-halocarbonyls, vinyl sulfones, alkene (thiol-ene coupling), alkyne (thiol-yne coupling), thiol (disulphide reaction) and other bioconjugating reagents known in the art. Probe reactive groups that react selectively with primary amines include but are not limited to succinimides, aldehydes, isocyanate, isothiocyanate, sulfonyl halides, sulfonates, aryl halides, imidoesters, alkyl halides or any other bioconjugating reagents known in the art. Probe reactive groups that could react with the tryptophan side chain include but are not limited to malondialdehydes and metallocarbenoids. Probe reactive groups that could react with the histidine side chain include but are not limited to epoxides, complexes with transition metals, and reagents suitable for histidine selective Michael addition. Probe reactive groups that could react with the tyrosine side chain include but are not limited to acetic anhydrides, N-acetylimidazoles, NHS esters, diazonium reagents, dicarboxylates, dicarboxamides and reagents suitable for Mannich-type reaction. Probe reactive groups that could react with the arginine side chain include but are not limited to phenylglyoxal, germinal diones and α-oxo-aldehydes. Probe reactive groups that could react with the aspartic and glutamic acid side chains include but are not limited to reagents suitable for carbodiimide-mediated activation. Probe reactive groups that could react with the methionine side chain include but are not limited to alkylating reagents of different structures in acidic condition. Probe reactive groups that could react with the α-amino groups at the N-terminal include but are not limited to acid anhydrides, acyl halogenides, ketenes, 2-pyridinecarboxyaldehydes and reagents suitable for transamination. Probe reactive groups that could react with serine and threonine at the N-terminal include but are not limited to reagents suitable for periodate oxidation and phosphate-assisted ligation. Probe reactive groups that could react with cysteine at the N-terminal include but are not limited to reagents suitable for native chemical ligation and thiazolidine-mediated ligation. Probe reactive groups that could react with tryptophan at the N-terminal include but are not limited to reagents suitable for sulfenylation-coupling and Pictet-Spengler reaction. Probe reactive groups that could react with histidine at the N-terminal include but are not limited to thiocarboxylic acid in the presence of Ellman's reagent. Probe reactive groups that could react with proline at the N-terminal include but are not limited to o-aminophenols and o-catechols in the presence of an oxidising agent. See also Koniev, et al., Chem Soc Rev. 2015 Aug. 7; 44(15):5495-551 for further details regarding bioconjugation of amino acids.

Probe reactive group that could be used for binding the target scaffold reactive group is basically the reverse as discussed above. In general, the reactive group at the side chain of the corresponding amino acid can be used as the probe reactive group. For example, thiol can be used as a probe reactive group for binding the scaffold reactive group of TBMB (i.e. alkyl bromide). Functional groups other than those present in the amino acid side chains but are known to react with the scaffold reactive group can also be used.

With regard to (3), the probe reactive group is preferably specific to the genetic display system. In some embodiments, the probe reactive group can be an antibody, a part of an antibody, or an antibody derivative which can target a specific antigen on the genetic display system. The target antigens may be expressed in the genetic display system which naturally, or expressed only when the genetic display system is transformed with the desired nucleic acids. In one embodiment, the target antigen can be any proteins, lipid or sugars present on the surface of the genetic display system. In one embodiment, the target antigen is a membrane protein of the genetic display system.

(ii) Probe Signalling Group

In the present specification, the terms “probe signalling group” and “signalling group” are used interchangeably.

In order to detect the presence of unconjugated peptide reactive groups or scaffold reactive groups, the probe must comprise or be linkable to a signalling group which gives a detectable signal directly or indirectly. Preferably, the signal can be quantified so that the amount or proportion of the corresponding unconjugated reactive groups can be measured.

In one embodiment, the signalling group provides a fluorescence signal upon light excitation. Fluorescent molecules are simple and advantageous as they respond directly and distinctly to light to produce a detectable signal. Moreover, fluorescent labels do not require additional reagents for detection. Fluorescent molecules suitable for biology are well known in the art (See, for example, Lavis, et al. ACS Chem Biol. 2008 Mar. 20; 3(3): 142-155; Herman B, Curr Protoc Cell Biol. 2001 May; Appendix 1:Appendix 1E; Christoph Greb, Fluorescent Dyes, Leica Microsystems, June 2012). Signalling groups can also comprise quantum dots or fluorescent proteins (See, for example, Bera, et al., Materials (Basel). 2010 April; 3(4): 2260-2345; Chudakov, et al., Physiol Rev. 2010 July; 90(3):1103-63.) In one embodiment, the fluorescent molecule is a donor for FRET, in which the emission spectrum of the fluorescent molecule overlaps with the absorption (or excitation) spectrum of another fluorescent molecule in proximity. In one embodiment, the probe signalling group comprises a combination of anthracene and rubrene, which is excited by light with a wavelength of around 340 nm and emits light detectable between 520-620 nm. In one embodiment, the probe signalling group comprises an Europium chelate which is excited at around 340 nm and emits light at around 615 nm.

In one embodiment, the probe signalling group gives luminescence such as chemiluminescence (see Dodeigne, et al. Talanta. 2000 Mar. 6; 51(3):415-39) and bioluminescence (see Paley, et al., Medchemcomm. 2014 Mar. 1; 5(3): 255-267; Aldo Roda, Chemiluminescence and Bioluminescence: Past, Present and Future (2011)). Chemiluminogenic labels include but are not limited to luminol, acridinium compounds, coelenterazine and analogues, thioxene derivatives, dioxetanes, systems based on peroxyoxalic acid and their derivatives. Luciferases for giving bioluminescence include but are not limited to firefly luciferase, chick beetle green, click beetle red, Lux AB and luciferase from Renilla reniformis, Gaussia princeps, Aequorea victoria and Vargula hilgendorfii. Luciferin for giving bioluminescence include but are not limited to D-luciferin, coelenterazine, vargulin and long chain aldehydes with FMN confactor.

In one embodiment, the probe signalling group comprises a photosensitiser. The photosensitiser can produce radicals or reactive oxygen species (or singlet oxygen) in the presence of a light source with appropriate wavelength. Photosensitisers include but are not limited to porphyrins, chlorins and dyes. Examples include but are not limited to aminolevulinic acid, silicon phthalocyanine Pc 4, m-tetrahydroxyphenylchlorin and mono-L-aspartyl chlorin e6. In one embodiment, the photosensitiser is phthalocyanine, which converts ambient oxygen to singlet oxygen upon illumination at about 680 nm. In one embodiment, the singlet oxygen can trigger a further reaction, such as chemiluminescence, in another probe signalling group present in the same probe or in a different probe in proximity.

In one embodiment, the probe signalling group provides a radioactive signal which can be detected by methods known in the art. Examples of radioisotopes include but are not limited to hydrogen-3, nitrogen-13, carbon-14, oxygen-15, fluorine-18, sodium-22, chlorine-36, sulphur-phosphorus-33, phosphorus-32, gallium-67, technetium-99m, iodine-123 and iodine-125.

In one embodiment, the probe signalling group comprises an enzyme or catalyst for catalysing a reaction to generate a detectable signal. Enzymes that can be used include but are not limited to horseradish peroxidase (HRP), alkaline phosphatase (AP), glucose oxidase and β-galactosidase, in which specific substrates are required for each enzyme (see Enzyme Probes, Pierce Protein Methods, ThermoFisher Scientific).

(iii) Linker

In one embodiment, both the probe reactive group and the signalling group are in the same probe and connected by a linker. The linker can be a spacer arm known in the art, such as poly(ethylene) glycol (PEG). It is known in the art that the number of repeats can affect the solubility of the probe. The skilled person has the knowledge to adjust the number of repeats to achieve the best result. The number of repeats can be 1-20, preferably 1-10, more preferably 1-5 and most preferably 2-3.

In one embodiment, the probe, which comprises the probe reactive group, is linkable to a signalling group. The linkage between the probe and the signalling group can comprise covalent bonding, hydrophilic interactions, hydrophobic interactions, van der Waals dispersion forces, dipole-dipole interactions and/or hydrogen bonding. In one embodiment, the probe comprises a biotin group and the signalling group comprises a streptavidin group. The probe reactive group can be linked to the biotin group by any linker known in the art, such as PEG.

(iv) Exposing the Peptide Ligand to a Probe

In order to measure the unconjugated reactive group on the peptide ligand displayed on the genetic display system, the genetic display system is first exposed to a probe. The conditions used in the present invention preferably comprise the following steps, all preferably conducted at room temperature:
1. Purified phage displaying the peptide ligand is neutralised and then diluted with assay buffer.
2. Probe solution is added to the phage.

Optionally, the following steps are performed if a reducing agent is required for efficient probe binding:
3. The probe-treated phage is mixed with resin equilibrated in assay buffer.
4. The resin is optionally washed with assay buffer.
5. The resin is incubated with reducing agent.
6. The resin is optionally washed with assay buffer.
7. Phage is eluted from the resin and then neutralised.

In step (1), the phage is neutralised with a buffer, preferably at pH 8.0. The neutralising buffer is preferably Tris-HCl. The buffer is preferably used at a concentration of 1 M.

In one embodiment, the unconjugated reactive group is the peptide reactive group. The assay buffer is preferably at pH 7.0. Suitable buffers include Tris, initially at pH 7.0. Alternative buffers may be used, including buffers with a pH in the physiological range, including NaHCO₃, NH₄CO₃ and HEPES. The Tris buffer is preferably used with sodium chloride. Preferably, the assay buffer is 25 mM Tris/150 μM NaCl at pH 7.0. The phage is preferably diluted half with the assay buffer.

In one embodiment, the unconjugated reactive group is the scaffold reactive group. The assay buffer is preferably at pH 8.0. The assay buffer is preferably degassed. Suitable buffers include NaHCO₃, initially at pH 8.0. Alternative buffers may be used, including buffers with a pH in the physiological range, including NH₄CO₃, HEPES and Tris-hydroxymethyl aminoethane, Tris, Tris-Acetate or MOPS. The NaHCO₃ buffer is preferably used at a concentration of 20 mM.

Preferably, the assay buffer does not contain EDTA. The phage is preferably diluted half with the assay buffer.

The concentration of the probe, temperature and time for step (2) are discussed herein.

The resin is preferably an ion exchange resin. Ion exchange resins are known in the art, and include any material suitable for anion exchange chromatography known in the art, such as an agarose based chromatography material, e.g. sepharoses like Fast Flow or Capto, polymeric synthetic material, e.g. a polymethacrylate such as Toyopearls, polystyrene/divinylbenzene, such as Poros, Source, or cellulose, e.g. Cellufine. In a preferred embodiment, the anion exchange resin material includes, but is not limited to a resin that carries a primary amine as ligand, e.g. aminohexyl sepharose, benzamidine sepharose, lysine sepharose, or arginine sepharose. In another preferred embodiment, the anion exchange resin material includes, but is not limited to a resin having a positively charged moiety at neutral pH, such as alkylaminoethane, like diethylaminoethane (DEAE), dimethylaminoethane (DMAE), or trimethylaminoethyl (TMAE), polyethyleneimine (PEI), quaternary aminoalkyl, quaternary aminoethane (QAE), quaternary ammonium (Q), and the like.

In step (5), reducing agent is added to a concentration of 1 mM. The concentration is for TCEP, and other values may apply to other reducing agents. Alternative reducing agents may be selected from dithiothreitol, thioglycolic acid, thiolactic acid, 3-mercaptopropionic acid, thiomalic acid, 2,3-dimercaptosuccinic acid, cysteine, N-glycyi-L-cysteine, L-cysteinylglycine and also esters and salts thereof, thioglycerol, cysteamine and C1-C4 acyl derivatives thereof, N-mesylcysteamine, Nacetylcysteine, N-mercaptoalkylamides of sugars such as N-(mercapto-2-ethyl) gluconamide, pantetheine, N-(mercaptoalkyl)-co-hydroxyalkylamides, for example those described in patent application EP-A-354 835, N-mono- or N,N-dialkylmercapto-4-butyramides, for example those described in patent application EP-A-368 763, aminomercaptoalkyl amides, for example those described in patent application EP-A-432 000, N-(mercaptoalkyl)succinamic acids and N-(mercaptoalkyl)succinimides, for example those described in patent application EP-A-465 342, alkylamine mercaptoalkyl amides, for example those described in patent application EP-A-514 282, the azeotropic mixture of 2-hydroxypropyl thioglycolate and of (2-hydroxy-1-methyl)ethyl thioglycolate as described in patent application FR-A-2 679 448, mercaptoalkylamino amides, for example those described in patent application FR-A-2 692 481, and N-mercaptoalkylalkanediamides, for example those described in patent application EP-A-653 202.

In step (7), the elution buffer is preferably at pH 5. Suitable buffers include citrate buffer, preferably containing sodium chloride. In one embodiment, the elution buffer is 50 mM citrate/1.5 M NaCl at pH 5.

In step (7), the phage is neutralised in a similar way as that of step (1).

In the situation that the peptide ligand is analysed by AlphaScreen, the probe mentioned above comprises the donor bead. The probe-treated phage is further diluted in AlphaScreen buffer (25 mM HEPES, 100 m M NaCl, 0.5% BSA, 0.05% Tween 20, 1 mM CaCl₂). The level of dilution depends on whether the phage is a single clone or a library, which can vary from 1 in 5 to 1 in 200, preferably 1 in 10 to 1 in 100. Preferably, a single clone phage sample is diluted at 1 in 100. Preferably, a single clone phage sample is diluted at 1 in 20 if the probe-bound phage has been treated with TCEP. Preferably, a phage library sample is diluted at 1 in 10. Once the phage is diluted, it is treated with the acceptor beads of AlphaScreen according to the standard protocol from PerkinElmer.

Additional steps can also be included in the procedure. Such steps are not mandatory, and do not significantly increase the yield or efficiency of the process.

For example, the reducing agent itself can be added in two steps; in a concentrated form, to effect reduction, and to maintain the displayed polypeptide in a reduced state.

The timing of the steps can also be varied, without significantly altering the efficiency of the procedure. For example, it has been found that reduction in TCEP for 20 minutes is as effective as reduction for 30 minutes.

(D) Determining the Extent of Cyclisation

(i) Measuring an Unconjugated Reactive Group on the Peptide Ligand

In the present invention, the peptide ligand comprises both peptide reactive groups and scaffold reactive groups. Measuring either one of the reactive groups which are not conjugated during the cyclisation reaction can allow the determination or estimation of the extent of cyclisation of the peptide ligand.

In one embodiment, the present invention uses AlphaScreen or AlphaLISA to measure the unconjugated reactive group. AlphaScreen and AlphaLISA assays require two bead types: Donor beads and Acceptor beads. Donor beads contain a photosensitizer (phthalocyanine) which converts ambient oxygen to singlet oxygen upon illumination at 680 nm. The singlet oxygen has a half-life of 4 pec and can diffuse approximately 200 nm in solution. If an Acceptor bead is within this distance, the singlet oxygen will transfer its energy to the thioxene derivatives within the Acceptor bead, subsequently emitting a light at 520-620 nm (AlphaScreen) or at 615 nm (AlphaLISA) for detection. In one embodiment, the Donor beads and the Acceptor beads are disposed on the peptide ligand and the genetic display system respectively.

Other measuring techniques for fluorescence (including FRET), luminescence and radioactivity as described herein are known in the art.

(ii) Measuring Both the Two Unconjugated Reactive Groups on the Peptide Ligand

The present invention further discloses a method in which both the peptide reactive groups and scaffold reactive groups are measured by repeating the method of (i) using a different probe. For example, the unconjugated peptide reactive groups on the peptide ligand are first measured using a probe binding to the peptide reactive groups. Next, the protocol is repeated to measure the unconjugated scaffold reactive groups on the peptide ligand using another probe which binds to the scaffold reactive groups.

Combining assay data from both probes could be used to verify the results of each other. This is particularly useful when the concentration of the molecular scaffold present is too low or too high, in which a false positive result would be obtained if only a single probe is used. For example, in the case of AlphaScreen, the signal is proportional to the amount of unconjugated reactive group present in the sample. When the concentration of molecular scaffold is low, it will be expected that the quantity of unconjugated scaffold reactive groups are also small. If only a single probe for binding unconjugated scaffold reactive group is used, a weak signal will be obtained. Nevertheless, this does not reflect the real situation as most of the peptide reactive groups are not conjugated. When the concentration of molecular scaffold is high, it will be expected that most of the peptide reactive groups are conjugated to molecular scaffolds. If only a single probe for binding unconjugated peptide reactive group is used, a weak signal will be obtained. However, this again does not reflect the real situation that the peptide reactive groups on the same polypeptide may not necessarily conjugate to a single molecular scaffold. A peptide ligand is only considered as correctly cyclised if the peptide reactive groups on a single polypeptide are conjugated to the scaffold reactive groups on a single molecular scaffold.

In one embodiment, the probe binding to unconjugated peptide reactive group is suitable for determining the minimum concentration of molecular scaffold for the cyclisation reaction, while the probe binding to unconjugated scaffold reactive group is suitable for determining the maximum concentration of molecular scaffold without artefact.

(iii) Optimising the Reaction Conditions for Cyclisation of Peptide Ligand Displayed on a Genetic Display System

The reaction conditions for cyclisation of peptide ligand can be optimised using the method disclosed in the present invention. The extent of cyclisation can be measured when the reaction is carried out using different parameters such as molecular scaffold concentration, temperature, buffer, pH, reaction time, type of reducing agent, reducing agent concentration, number of washing and type of purification resin. In one embodiment, the condition which gives the weakest signal from both the two probes (as mentioned in (ii)) can be chosen.

The method can also be used for comparing the extent of cyclisation of different molecular scaffolds. This can assist the screening of better molecular scaffolds, as a molecular scaffold with better cyclisation ability can increase the yield of peptide ligand and facilitate the screening of peptide ligands as drugs.

(iv) Screening of Clones with Correct Cyclisation

The present invention also allows the screening of clones with correct cyclisation. Even if the same molecular scaffold and the optimised reaction condition are used, the cyclisation efficiency of different polypeptides in a library of genetic display system can be different. This is particularly crucial when several peptide ligands with similar binding activity to a target (such as an antigen of a bacteria, virus or cancer cell) are obtained. The screening of correct cyclisation allows the selection of the best peptide ligand having high production yield.

(E) Use of Polypeptide Ligands According to the Invention

Peptide ligands of the present invention may be employed in in vivo therapeutic and prophylactic applications, in vitro and in vivo diagnostic applications, in vitro assay and reagent applications, and the like. Ligands having selected levels of specificity are useful in applications which involve testing in non-human animals, where cross-reactivity is desirable, or in diagnostic applications, where cross-reactivity with homologues or paralogues needs to be carefully controlled. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

The peptide ligands of the present invention will typically find use in preventing, suppressing or treating inflammatory states, allergic hypersensitivity, cancer, bacterial or viral infection, and autoimmune disorders (which include, but are not limited to, Type I diabetes, multiple sclerosis, rheumatoid arthritis, systemic lupus erythematosus, Crohn's disease and myasthenia gravis).

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

Generally, the present peptide ligands will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cylcosporine, methotrexate, adriamycin or cisplatinum, and immunotoxins. Pharmaceutical compositions can include “cocktails” of various cytotoxic or other agents in conjunction with the selected antibodies, receptors or binding proteins thereof of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that use levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

(F) Mutation of Polypeptides

The desired diversity is typically generated by varying the selected molecule at one or more positions. The positions to be changed are selected, such that libraries are constructed for each individual position in the loop sequences. Where appropriate, one or more positions may be omitted from the selection procedure, for instance if it becomes apparent that those positions are not available for mutation without loss of activity.

The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The same techniques could be used in the context of the present invention. For example, the H₃ region of a human tetanus toxoid-binding Fab has been randomised to create a range of new binding specificities (Barbas et al. (1992) Proc. Natl. Acad. Sci. USA, 89: 4457). Random or semi-random H₃ and L3 regions have been appended to germline V gene segments to produce large libraries with mutated framework regions (Hoogenboom-& Winter (1992) R Mol. Biol., 227: 381; Barbas et al. (1992) Proc. Natl. Acad. Sci. USA, 89: 4457; Nissim et al. (1994) EMBO J, 13: 692; Griffiths et al. (1994) EMBO J, 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) BiolTechnology, 13: 475; Morphosys, WO 97/08320, supra).

However, since the polypeptides used in the present invention are much smaller than antibodies, the preferred method is to synthesise mutant polypeptides de novo. Mutagenesis of structured polypeptides is described above, in connection with library construction.

The invention is further described below with reference to the following examples.

### Examples

Unless otherwise stated, any methods and materials similar or equivalent to those described herein can be used in the practice or testing of the present invention. Methods, devices, and materials suitable for such uses are described above. All publications cited herein are incorporated herein by reference in their entirety for the purpose of describing and disclosing the methodologies, reagents, and tools reported in the publications that might be used in connection with the invention.

### Example 1: Optimising Probe Concentration for the Peptide-Reactive Probe Assay

Background

An assay was developed for qualitative analysis of the degree of cyclisation by scaffolds on peptides presented by phage. Here, a biotinylated maleimide probe was used to measure the free thiols on peptides where cyclisation has not occurred. The concentration of peptide-reactive probe which gave the optimum signal was identified. One sample each of unmodified and iodoacetamide-capped phage was assayed alongside any scaffolded samples as positive and negative controls respectively.

Aim

To optimise the concentration of peptide-reactive probe required for a single clone and a library of peptide ligands on phage display.

Materials and Methods

(A) Phage Modification

Details of the modification protocol are disclosed in WO 2014/140342.

The modification buffer (20 mM NaHCO₃, 5 mM EDTA) was prepared and degassed.

For assaying single clones, phage were amplified via infection and overnight growth in TGI E. coli. Phage were then modified with the appropriate scaffold using either the Kingfisher Duo, mL or Flex liquid handling systems. When assaying phage libraries, a sample of the TE/Ethylene Glycol store was diluted at 100 fold in modification buffer prior to modification.

An unmodified and iodoacetamide capped samples were run alongside scaffold samples as positive and negative controls respectively.

The SuporQ beads were prepared by:
(1) washing 25 μL beads per sample for three times in 1 M NaHCO₃(pH of stock >8.5);
(2) resuspending the beads in original volume in 1 M NaHCO₃
(3) adding 1.25 μL 1 M TCEP for every 25 μL SuporQ beads.

Washing buffer: for single clones only, 1 mL of 1 μM TCEP in modification buffer was prepared per sample plus 1 mL dead volume.

Iodoacetamide solution: 1 mL of 10 μM iodoacetamide (plus 1 mL dead volume) was prepared and diluted at 1 in 5 in modification buffer.

Scaffold solution: 1 mL of molecular scaffold (plus 1 mL dead volume) was prepared per sample and diluted at 1 in 5 in modification buffer to an appropriate final concentration with 20% acetonitrile when TBMB and TATA were used as the molecular scaffold with final concentrations of 60 μM and 400 μM respectively.

33 μL of washed beads with TCEP were combined with 1 mL of overnight culture containing the phage. For single clones, 1 mL of overnight culture was used. For libraries, 1 in 100 dilution of library in modification buffer was used. If more than one library format is assayed, the sample libraries are diluted to an equivalent phage titre in 50:50 TE/ethylene glycol prior to 1 in 100 dilution in modification buffer. The samples were mixed by rotation for 20 minutes. The samples were centrifuged at 3000 rpm for one minute and the supernatant was carefully removed.

For single clones, 1 mL of washing buffer was added to resuspend the resin whilst washing away the majority of the any remaining TCEP prior to the addition of the molecular scaffold. The samples were centrifuged as before and the supernatant was carefully removed.
For modification: 1 mL of scaffold solution was added to each sample
For positive control: 1 mL of modification buffer was added
For negative control: 1 mL of iodoacetamide solution was added

The samples were mixed by rotation for 10 minutes. The samples were centrifuged as before and the supernatant was carefully removed. 1 mL of modification buffer was added. The samples were centrifuged as before and the supernatant was carefully removed. 50 μL of elution buffer (50 mM citrate, 1.5 M NaCl, pH 5.0) was added to each sample and the samples were mixed for minutes on a shaking platform. Each sample was spun at 13000 rpm in a microfuge for one minute before the supernatant was carefully removed and retained. The supernatant was recentrifuged, to remove any remaining traces of the resin, and the supernatant was carefully removed and retained.

For libraries, 1 mL of modification buffer was added to resuspend the resin whilst washing away the majority of the any remaining TCEP prior to the addition of the molecular scaffold. The samples were centrifuged as before and the supernatant was carefully removed.
For modification: 1 mL of scaffold solution was added to each sample
For positive control or pre-modified library phage: 1 mL of modification buffer was added
For negative control: 1 mL of iodoacetamide solution was added

The samples were mixed by rotation for 10 minutes. The samples were centrifuged as before and the supernatant was carefully removed. 1 mL of modification buffer was added. The samples were centrifuged as before and the supernatant was carefully removed. 50 μL of elution buffer (50 mM citrate, 1.5 M NaCl, pH 5.0) was added to each sample and the samples were mixed for minutes on a shaking platform. Each sample was spun at 13000 rpm in a microfuge for one minute before the supernatant was carefully removed and retained. The supernatant was recentrifuged, to remove any remaining traces of the resin, and the supernatant was carefully removed and retained.

The above procedures can be performed automatically using Kingfisher with a pre-set programme.

The phage was neutralised with 10 μL of 1 M Tris-HCl/pH 8.0 per well. Each phage sample was diluted at 50:50 in the assay buffer (25 mM Tris, 150 μM NaCl, pH 7.0) by adding 60 μL assay buffer to 60 μL neutralised phage.

(B) Preparation and Addition of Probe

A stock solution of Maleimide-PEG2-Biotin probe was prepared by solubilising powder in PBS to give a concentration of 20 mM. 20 μL of probe solution (plus 20 μL dead volume) was prepared for each sample by diluting the stock in assay buffer. A series of probe solution having different concentrations was prepared for optimising the protocol. 20 μL of each phage sample was added to 20 μL of probe solution, mixed, sealed, and incubated at room temperature for 2 hours.

(C) AlphaScreen

For single clone phage, the probe-bound phage was diluted at 1 in 100 to 200 μL in AlphaScreen buffer (25 mM HEPES, 100 mM NaCl, 0.5% BSA, 0.05% Tween20, 1 mM CaCl₂, pH 7.4). For library phage, the probe-bound phage was diluted at 1 in 10 to 200 μL in AlphaScreen buffer. 15 μL of phage sample was added to Perkin Elmer Opti 384 plate. Under subdued lighting, the AlphaScreen acceptor beads were vortexed and diluted to 1 in 66 in AlphaScreen buffer. 5 μL of the diluted AlphaScreen acceptor beads was added to each well. The plate was sealed and incubated for 30 minutes at room temperature in the dark. Under subdued lighting, the AlphaScreen streptavidin donor beads were vortexed and diluted to 1 in 50 in AlphaScreen buffer. 5 μL of the diluted AlphaScreen streptavidin donor beads was added to each well. The plate was sealed and incubated for 1 hour at room temperature in dark. The plate was then read on Pherastar and the fluorescence signal from each well was measured.

Results

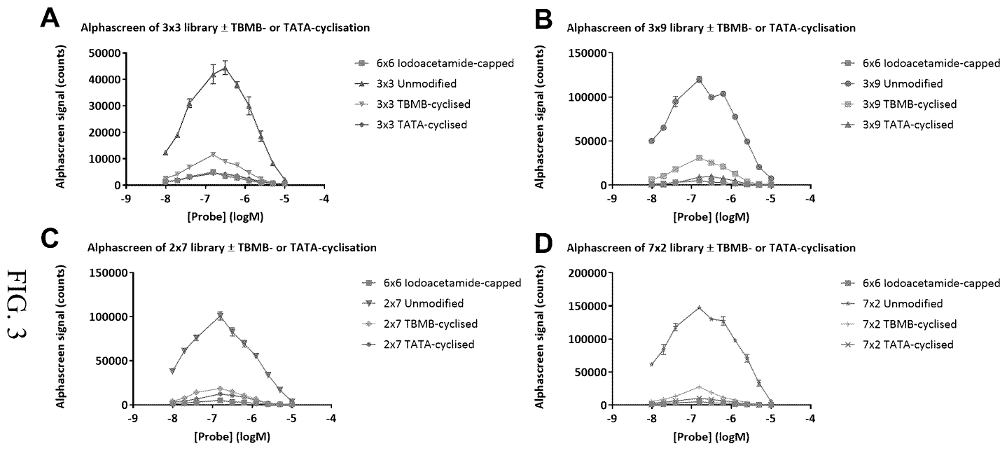

FIG. 2 shows the result of using different probe concentration for the single clone 17-88 (displaying a polypeptide of SEQ ID NO:1). The positive control gave a strong signal, indicating that free cysteine residues were present on the polypeptide. The negative control (in which all the cysteine residues were capped by iodoacetamide) gave a weak signal, showing that the signal of the assay was not affected by other the presence of other groups. The TBMB cyclised sample also gave a weak signal, indicating that most of the cysteine residues on the polypeptide were conjugated to the molecular scaffold. The assay was also repeatable as shown by FIG. 2. The optimum probe concentration for single clones was determined to be 2.5 μM.

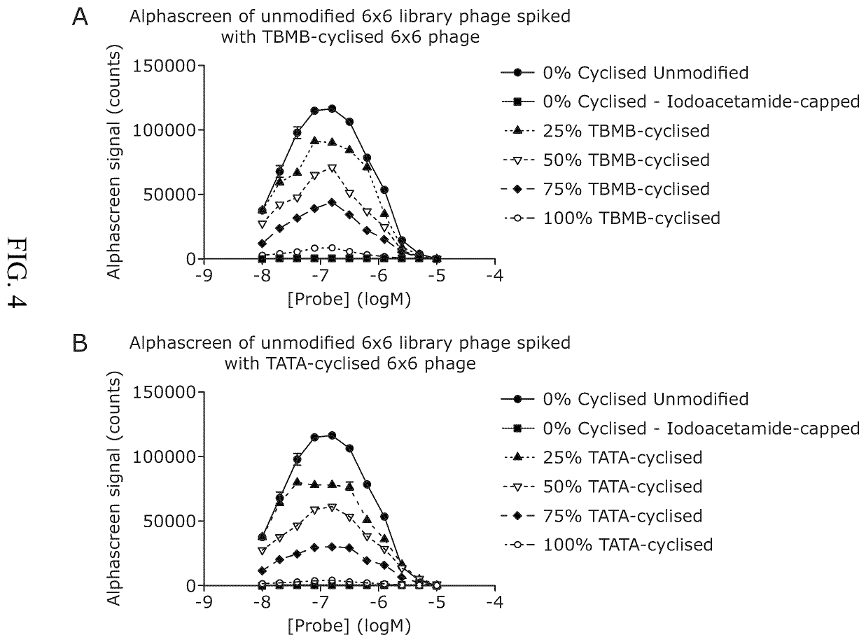

FIGS. 3A-3D show the result of using different probe concentration of different phage libraries. Similar to the results obtained from single clones, the positive and negative controls for the libraries also gave a strong and a weak signal respectively, showing that the assay can also be applied to phage libraries. The optimum probe concentration varied slightly in libraries but a good window was seen at around 100 nM probe. In general, higher background signal was seen in libraries. This is likely because libraries contain some phage that present peptides with greater or fewer than three cysteine residues that do not cyclise correctly, and a signal is seen when the probe binds to these residues.

### Example 2: Qualifying Cyclisation of Libraries with the Peptide-Reactive Probe Assay

Background

The results from Example 1 showed that the peptide-reactive probe assay could allow the detection of cyclisation of the peptide ligand. Nevertheless, it is crucial to understand if the assay can provide an estimation of the extent of cyclisation. Here, library samples with different levels of cyclisation were assayed using the biotinylated maleimide probe of Example 1.

Aim

To test if the peptide-reactive probe assay can be used for determining the extent of cyclisation of peptide ligands.

Materials and Methods

6×6 phage library samples were combined in different ratios of unmodified:cyclised phage and assayed using the protocol of Example 1. Relative amounts of uncyclised phage were detected and ranked.

Results

From FIGS. 4A-4B, it is clear that the signal obtained from the assay is reversely proportional to the percentage of cyclised phage. This demonstrates that the peptide-reactive probe assay can be used for determining or estimating the extent of cyclisation of peptide ligands.

### Example 3: Optimising the Scaffolding Conditions on Single Clones and Libraries Using the Peptide-Reactive Probe Assay

Background and Aim

As the protocol of the assay of Example 1 had been optimised, it could be used for optimising the cyclisation reaction of peptide ligand. Here, the signal obtained from samples treated with different concentrations of molecular scaffolds were analysed to select the optimised concentration.

Materials and Methods

The same protocol of Example 1 was used for single clones. Four combinations of single clones and molecular scaffolds were assayed:
1) 17-88 phage (displaying polypeptide of SEQ ID NO:1)+TBMB (12.5 μM, 25 μM, 50 μM, 60 μM, 100 μM, 200 μM)
2) 55-28-00 phage (displaying polypeptide of SEQ ID NO:2)+TATA (25 μM, 50 μM, 100 μM, 200 μM, 400 μM, 800 μM, 1600 μM)
3) 06-663-00 phage (displaying polypeptide of SEQ ID NO:3)+TCAZ (25 μM, 50 μM, 100 μM, 200 μM, 400 μM, 800 μM, 1600 μM)
4) 17-69-07 phage (displaying polypeptide of SEQ ID NO:4)+TCCU (25 μM, 50 μM, 100 μM, 200 μM, 400 μM, 800 μM, 1600 μM)

The same protocol of Example 1 was used for libraries, except that only the signal obtained by optimised probe concentration (100 nM) was measured. Five different libraries (6×6, 3×3, 3×9, 2×7, 7×2) treated with four different molecular scaffolds (60 μM TBMB, 400 μM TATA, 400 μM TCAZ, 400 μM TCCU) were assayed. The concentrations of the molecular scaffold used were based on the results obtained from the single clones.

Results

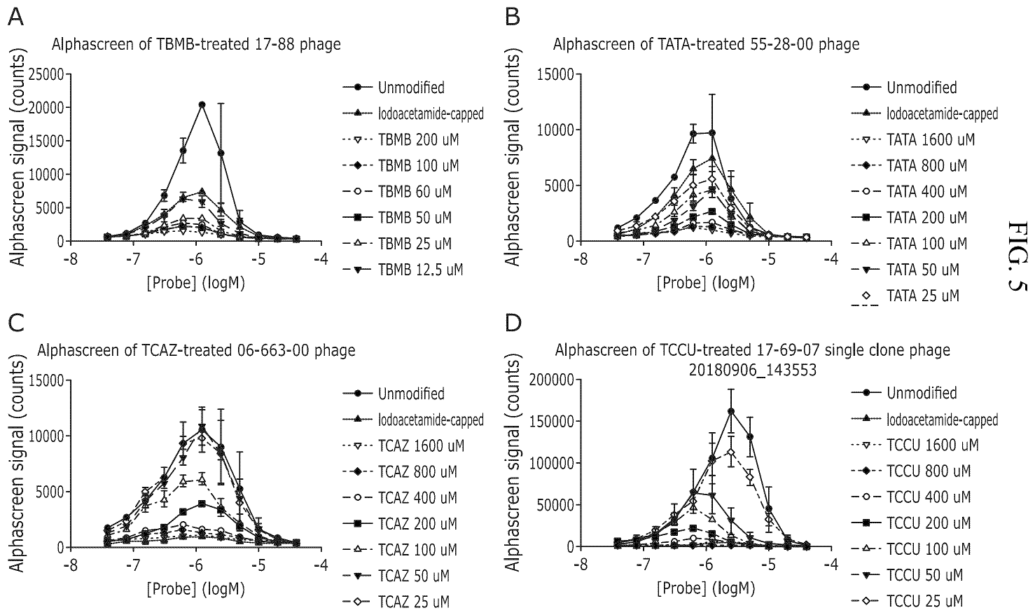

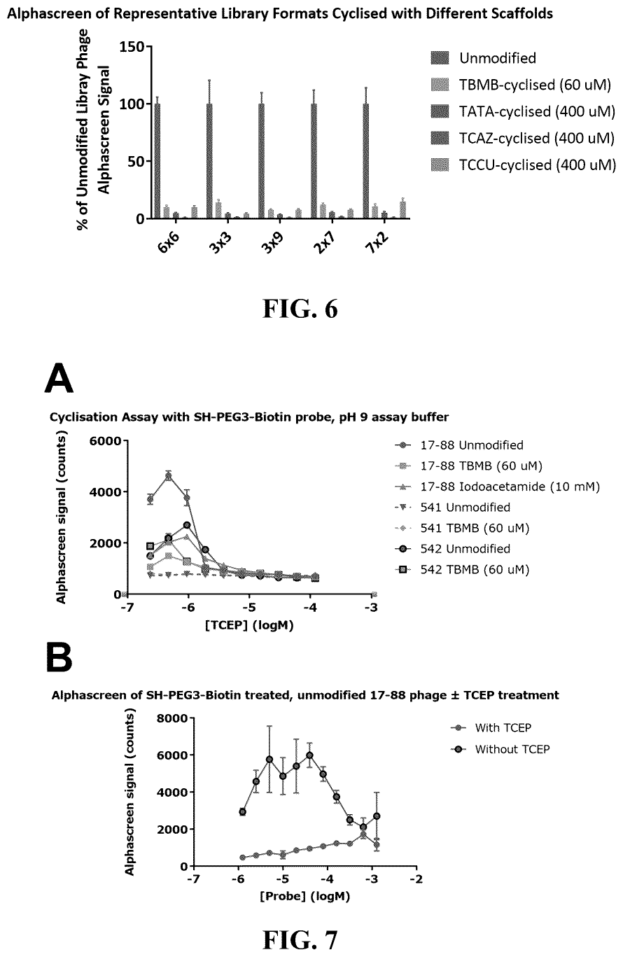

FIGS. 5A-5D show the results for the different combinations of single clones and molecular scaffolds. The results clearly demonstrated that the extent of cyclisation of the peptide ligand was dependent on the concentration of molecular scaffold used for the reaction. The optimum concentrations for TBMB, TATA, TCAZ and TCCU were determined to be ≥60 μM, ≥400 μM, ≥400 μM, and ≥400 μM respectively. FIG. 6 shows that the scaffolds can cyclise a range of library formats at the optimum concentrations obtained from the assays of single clones.

### Example 4: Optimising TCEP Concentration for the Scaffold-Reactive Probe Assay

Background

A further assay was developed for qualitative analysis of the degree of cyclisation by scaffolds on peptides presented by phage. Here, a biotinylated thiol probe was used to measure the free scaffold groups on peptides where incomplete/incorrect cyclisation has occurred, allowing for qualitative analysis of the degree of cyclisation of peptides presented by phage. A major problem of using a thiol probe is that it can bind to free thiols on the polypeptide (i.e. disulphide formation) as well as the free scaffold groups (e.g. TBMB). In this Example, it was demonstrated that the addition of TCEP can solve the above problem.

Aim

To optimise the concentration of TCEP added so as to eliminate the background signal from the polypeptides without inhibiting the assay signal.

Materials and Methods

(A) Phage Modification

The modification protocol is the same as that of Example 1 of the present specification, except that the assay buffer was 20 mM NaHCO₃ (without EDTA).

(B) Preparation and Addition of Probe

A stock solution of SH-PEG3-Biotin probe was prepared by solubilising powder in AcN/H₂O to give a concentration of 20 mM. 20 μL of probe solution (plus 20 μL dead volume) was prepared for each sample by diluting the stock in assay buffer. The probe concentrations used for single clones and libraries were 320 μM and 1280 μM respectively. 20 μL of each phage sample was added to 204 of probe solution, mixed, sealed, and incubated at room temperature for 1 hour.

(C) TCEP Treatment

Each sample was diluted with 4274 assay buffer and mixed with 334 SuporQ beads prepared as before in the modification process. 1 mL of assay buffer was added to resuspend the resin. The samples were centrifuged as before and the supernatant was carefully removed. The samples were incubated with 1 mL of TCEP of different concentrations in assay buffer for 30 minutes. The samples were centrifuged as before and the supernatant was carefully removed. 1 mL of assay buffer was added to resuspend the resin. The samples were centrifuged as before and the supernatant was carefully removed. 504 of elution buffer (50 mM citrate, 1.5 M NaCl, pH 5.0) was added to each sample and the samples were mixed for 5 minutes on a shaking platform. Each sample was spun at 13000 rpm in a microfuge for one minute before the supernatant was carefully removed and retained. The supernatant was recentrifuged, to remove any remaining traces of the resin, and the supernatant was carefully removed and retained. The above procedures can be performed automatically using Kingfisher with a pre-set programme. The phage was then neutralised with 10 μL of 1 M Tris-HCl/pH 8.0 per well.

(D) AlphaScreen

The protocol for AlphaScreen is identical to that of Example 1.

Results

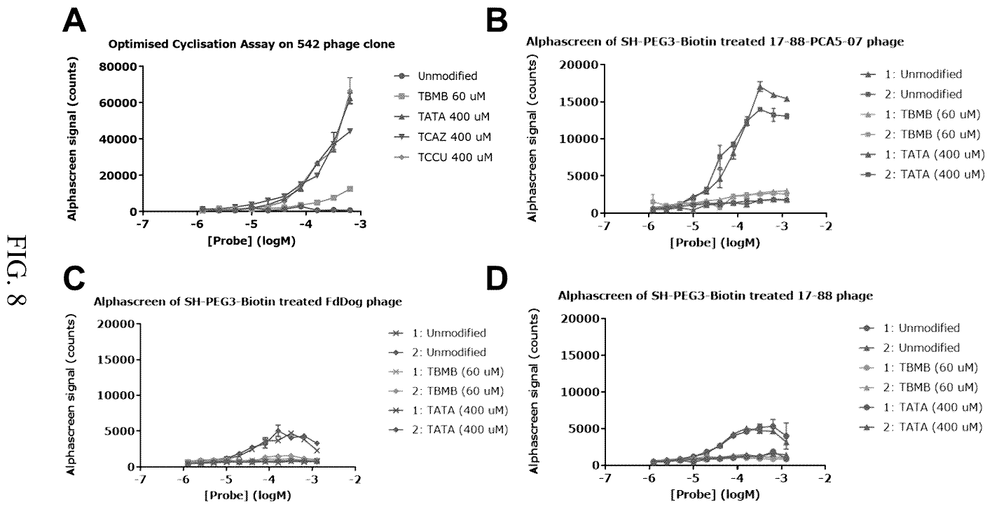

As revealed in FIG. 7A, the addition of TCEP inhibited assay signal at high concentrations, but was required for preventing non-specific binding of the probe. The single phage clones 541 and 542 were used as positive controls as their displayed polypeptides (SEQ ID NOs: 5 and 6) have less than 3 cysteine residues, which cannot be correctly cyclised with TBMB. In general, the AlphaScreen reagents were compatible with less than 10 mM TCEP. FIG. 7B demonstrates the difference of signal of the unmodified phage in the presence or absence of TCEP when different probe concentrations were used, which again reveals the importance of TCEP.

### Example 5: Optimising Probe Concentration for the Scaffold-Reactive Probe Assay

Aim

To optimise the concentration of scaffold-reactive probe required for a single clone on phage display.

Materials and Methods

The protocol is identical to that of Example 4, except that the phage was treated with different probe concentrations, and the probe-bound phage was treated with 1 mM TCEP. The concentrations of TBMB, TATA, TCAZ and TCCU used were 60 μM, 400 μM, 400 μM and 400 μM respectively.

Results

FIGS. 8A-8B show the results of using different probe concentration for two positive control single phage clones 542 and 17-88-PCA5, in which their displayed polypeptides (SEQ ID NOs: 6 and 7) have less than 3 cysteine residues. FIG. 8C shows the results of using different probe concentration for the negative control FdDog phage which does not present any polypeptides with cysteine residues. FIG. 8D shows the results of using different probe concentration for the single clone 17-88 phage. The results were repeatable as shown by FIGS. 8B-8D. The negative controls gave a reliably low signal, showing that the signal of the assay was not affected by other the presence of other groups. However, signals seen in the positive controls varied significantly. The optimum probe concentration for single clones was determined to be 320 04.

### Example 6: Qualifying Cyclisation of Libraries with the Scaffold-Reactive Probe Assay

Background

The results from Example 5 showed that the peptide-reactive probe assay could allow the detection of cyclisation of the peptide ligand. Nevertheless, it is crucial to understand if the assay can provide an estimation of the unconjugated scaffold reactive groups. Here, single clone samples with different levels of cyclisation were assayed using the biotinylated thiol probe of Example 4.

Aim

To test if the scaffold-reactive probe assay can be used for measuring the unconjugated scaffold reactive groups.

Materials and Methods

The 542 and 17-88 single clone samples were each combined in different ratios of unmodified:cyclised phage and assayed using the protocol of Example 4. The probe-bound phage was treated with 1 mM TCEP. Relative amounts of uncyclised phage were detected and ranked.

Results

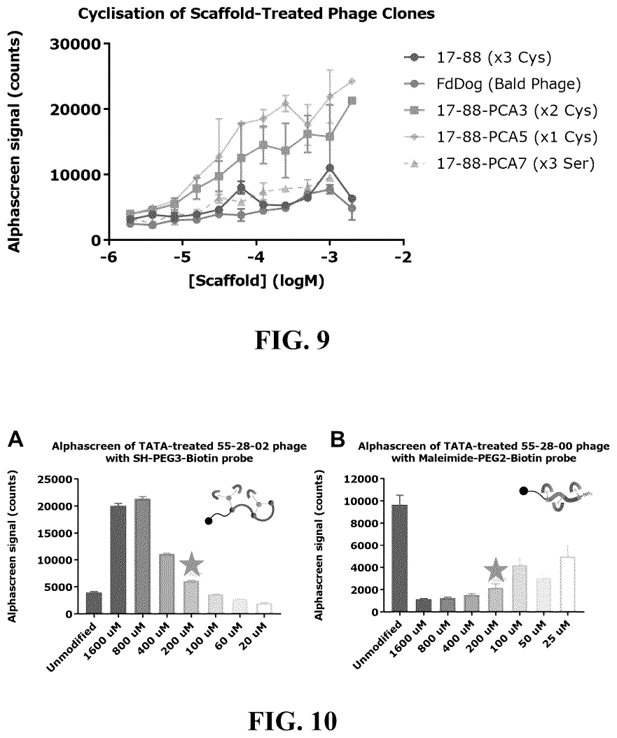

From FIGS. 9, it is clear that the signal obtained from the assay for the positive control (542 clone) is proportional to the percentage of TATA-modified clones. The TATA-modified 542 clone gave signals because the polypeptides displayed on the positive control cannot undergo correct cyclisation. The TBMB-modified 17-88 clone did not give any signal as the displayed polypeptides were correctly cyclised. The results demonstrated that the scaffold-reactive probe assay can be used for determining or estimating the unconjugated scaffold reactive groups on the peptide ligands.

### Example 7: Screening of Clones with Correct Cyclisation Using the Scaffold-Reactive Probe Assay

Background

The results from Example 6 showed that the scaffold-reactive probe assay can potentially be used as a tool to distinguish polypeptides that can be correctly cyclised with those that cannot. It would be interesting to know if it can further compare the cyclisation efficiency for polypeptides having 3 cysteine residues, which should all undergo correct cyclisation in theory. Here, the assay was applied to a number of single clones displaying polypeptides with 0-3 cysteine residues in order to select the clones with the best cyclisation efficiency.

Aim

To test if the scaffold-reactive probe assay can be used for screening clones with efficient cyclisation.

Materials and Methods

A number of single clones were treated with TBMB or TCAZ and assayed using the protocol of Example 4. The probe-bound phage was treated with 1 mM TCEP.

Results

FIG. 9 clearly shows that non-bicycle clones gave greater signal than bicycle, bald, or triple serine phage clones. FIG. 11 further shows a number of clones displaying 1-3 cysteine residues. The signal:background (S:B) ratio was calculated based on the ratio of the signal obtained from TCAZ-modified phage to the signal obtained from unmodified phage. A high S:B ratio indicates that the polypeptide is not correctly cyclised. It is clear that polypeptides with 3 cysteine residues generally have a lower S:B ratio than those having less than 3 cysteine residues. Surprisingly, the cyclisation efficiency for polypeptides having 3 cysteine residues can be further distinguished. In particular, the clones P-085-071_B12, P-085-071_D06, P-08-071_D08 and P-085-071_G05 showed poor cyclisation even they displayed polypeptides with 3 cysteine residues.

### Example 8: Optimising the Scaffolding Conditions on Single Clones by Combining the Peptide-Reactive Probe Assay and the Scaffold-Reactive Probe Assay

Background and Aim

As the protocol of the scaffold-reactive probe assay of Example 4 had been optimised, it could be used for optimising the cyclisation reaction of peptide ligand. To test the optimised concentration of molecular scaffold required for effective cyclisation, the signal obtained from samples treated with different concentrations of molecular scaffolds was analysed.

The problem of using only one assay for optimising the cyclisation reaction is that false positive results may be obtained. For the peptide-reactive probe assay, a weak signal would be obtained when a high concentration of molecular scaffold is used, but this does not necessarily indicate that the polypeptide is correctly cyclised, as a single polypeptide can be conjugated to more than one molecular scaffolds. On the other hand, a weak signal would be obtained from the scaffold-reactive probe assay if a low concentration of molecular scaffold is used, as the number of unconjugated scaffold reactive groups is low, but this does not indicate that all the polypeptides are conjugated with molecular scaffolds. Here, it was demonstrated that a combination of the peptide-reactive probe assay and the scaffold-reactive probe assay can be used for optimising the cyclisation reaction of peptide ligand.

Materials and Methods

The peptide-reactive probe assay of Example 1 was used. The 55-28-00 phage was treated with different concentrations of TATA (25 μM, 50 μM, 100 μM, 200 μM, 400 μM, 800 μM, 1600 μM). The probe concentration used was 2.5 μM.

The scaffold-reactive probe assay of Example 4 was used. The 55-28-02 (displaying polypeptide of SEQ ID NO:28) phage was treated with different concentrations of TATA (20 μM, 60 μM, 100 μM, 200 μM, 400 μM, 800 μM, 1600 μM). The probe concentration used was 320 μM. The probe-bound phage was treated with 1 mM TCEP.

Results

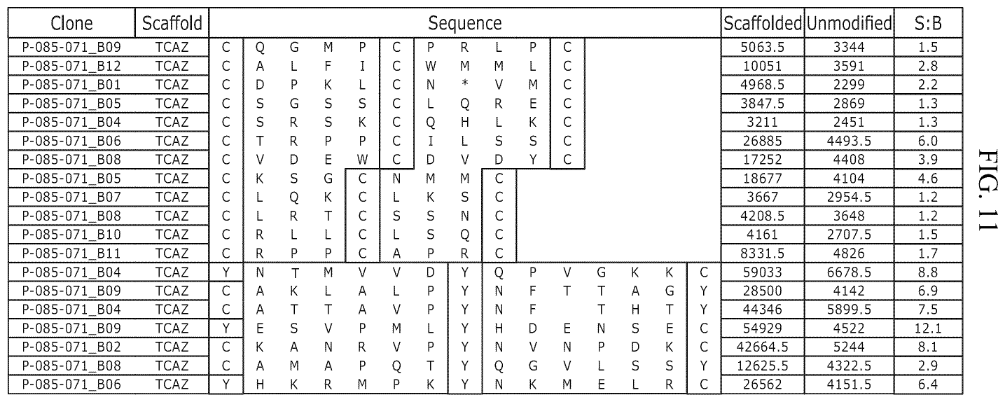

FIGS. 10A-10B show that the optimum concentration for the molecular scaffold is 200 μM. The combination of the results from the two assays allows the determination of the extent of cyclisation of the peptide ligand, and at the same time ensures that most of the polypeptides are conjugated to a single molecular scaffold.

SEQUENCES

SEQ ID NO: 1 (Polypeptide displayed by phage 17-88)
CPYSWETCLF GDYRC
SEQ ID NO: 2 (Polypeptide displayed by phage 55-28-00)
CPLVNPLCLT SGWKC
SEQ ID NO: 3 (Polypeptide displayed by phage 06-663-00)
CGHVAPWCWR TNHDC
SEQ ID NO: 4 (Polypeptide displayed by phage 17-69-07)
CYNEFGCEDF YDIC
SEQ ID NO: 5 (Polypeptide displayed by phage 541)
SGTGAASTGG ATC
SEQ ID NO: 6 (Polypeptide displayed by phage 17-88-PCA5)
CPYSWETSLF GDYRS
SEQ ID NO: 7 (Polypeptide displayed by phage 17-88-PCA3)
CPYSWETCLF GDYRS
SEQ ID NO: 8 (Polypeptide displayed by phage 17-88-PCA7)
SPYSWETSLF GDYRS
SEQ ID NO: 9 (Polypeptide displayed by phage P-085-071_B09)
CQGMPCPRLP C
SEQ ID NO: 10 (Polypeptide displayed by phage P-085-071_B12)
CALFICWMML C
SEQ ID NO: 11 (Polypeptide displayed by phage P-085-071_C01)
CDPKLCN*VM C
SEQ ID NO: 12 (Polypeptide displayed by phage P-085-071_C05)
CSGSSCLQRE C
SEQ ID NO: 13 (Polypeptide displayed by phage P-085-071_D04)
CSRSKCQHLK C
SEQ ID NO: 14 (Polypeptide displayed by phage P-085-071_D06)
CTRPPCILSS C
SEQ ID NO: 15 (Polypeptide displayed by phage P-085-071_D08)
CVDEWCDVDY C
SEQ ID NO: 16 (Polypeptide displayed by phage P-085-071_G05)
CKSGCNMMC
SEQ ID NO: 17 (Polypeptide displayed by phage P-085-071_G07)
CLQKCLKSC
SEQ ID NO: 18 (Polypeptide displayed by phage P-085-071_G08)
CLRTCSSNC
SEQ ID NO: 19 (Polypeptide displayed by phage P-085-071_G10)
CRLLCLSQC
SEQ ID NO: 20 (Polypeptide displayed by phage P-085-071_G11)
CRPPCAPRC
SEQ ID NO: 21 (Polypeptide displayed by phage P-055-173_B04)
YNTMVVDYQP VGKKC
SEQ ID NO: 22 (Polypeptide displayed by phage P-055-173_E09)
CAKLALPYNF TTAGY
SEQ ID NO: 23 (Polypeptide displayed by phage P-055-173_E04)
CATTAVPYNF HTHTY
SEQ ID NO: 24 (Polypeptide displayed by phage P-055-173_D09)
YESVPMLYHD ENSEC
SEQ ID NO: 25 (Polypeptide displayed by phage P-055-173_C02)
CKANRVPYNV NPDKC
SEQ ID NO: 26 (Polypeptide displayed by phage P-055-173_E08)
CAMAPQTYQG VLSSY
SEQ ID NO: 27 (Polypeptide displayed by phage P-055-173_D06)
YHKRMPKYNK MELRC
SEQ ID NO: 28 (Polypeptide displayed by phage 55-28-02)
CPMVNPLCLH PGWIC

1. A method for determining an extent of cyclisation of a peptide ligand displayed on a genetic display system, wherein the peptide ligand comprises a polypeptide covalently linked to a molecular scaffold at two or more amino acid residues, comprising the steps of:
(a) exposing the polypeptide displayed on the genetic display system to the molecular scaffold, wherein said polypeptide comprises two or more peptide reactive groups on said two or more amino acid residues which form covalent bonds with the molecular scaffold at two or more scaffold reactive groups, to give the peptide ligand;
(b) removing unreacted molecular scaffold from the genetic display system;
(c) exposing the peptide ligand displayed on the genetic display system to a first probe, wherein the first probe binds to a first unconjugated reactive group on the peptide ligand; and
(d) measuring the first unconjugated reactive group on the peptide ligand.

2. The method according to claim 1, wherein the first probe comprises or is linkable to a first signalling group, the first signalling group produces a first signal directly or indirectly to indicate the first unconjugated reactive group on the peptide ligand.

3. The method according to claim 2, further comprising exposing the peptide ligand displayed on the genetic display system to a second probe after step (c), wherein the second probe binds to the genetic display system, and comprises or is linkable to a second signalling group.

4. The method according to claim 3, wherein the second signalling group is triggered by the first signal to produce a second signal.

5. The method according to claim 3, wherein the second signalling group produces a second signal, the second signal triggering the first signalling group to produce the first signal.

6. The method according to claim 4, wherein the first signalling group comprises a first photosensitiser configured to convert ambient oxygen molecules to singlet oxygen molecules, and the second signalling group comprises a first chemiluminescent molecule configured to be excited by singlet oxygen molecules.

7. The method according to claim 6, wherein the second signalling group further comprises a first fluorescent group, the first fluorescent group is configured to be excited by chemiluminescence of the first chemiluminescent molecule.

8. The method according to any of claims 1-7, wherein the first unconjugated reactive group is one of the two or more peptide reactive groups.

9. The method according to any of claims 1-7, wherein the first unconjugated reactive group is one of the two or more scaffold reactive groups.

10. The method according to claim 9, wherein step (c) further comprises treating the genetic display system with a reducing agent after exposing the genetic display system to the first probe.

11. The method according to claim 10, wherein the reducing agent is TCEP.

12. The method according to any of claims 9-11, wherein the method is further repeated by using a third probe in step (c), the third probe binds to a second unconjugated reactive group, wherein the second unconjugated reactive group is one of the two or more peptide reactive groups.

13. The method according to claim 12, wherein the third probe comprises or is linkable to a third signalling group, the third signalling group produces a third signal directly or indirectly to indicate the second unconjugated reactive group on the peptide ligand.

14. The method according to claim 13, further comprising exposing the peptide ligand displayed on the genetic display system to a fourth probe after using the third probe in step (c), wherein the fourth probe binds to the genetic display system, and comprises or is linkable to a fourth signalling group.

15. The method according to claim 14, wherein the fourth signalling group is triggered by the third signal to produce a fourth signal.

16. The method according to claim 14, wherein the fourth signalling group produces a fourth signal, the fourth signal triggering the third signalling group to produce the third signal.

17. The method according to claim 15, wherein the third signalling group comprises a second photosensitiser configured to convert ambient oxygen molecules to singlet oxygen molecules, and the fourth signalling group comprises a second chemiluminescent molecule configured to be excited by singlet oxygen molecules.

18. The method according to claim 17, wherein the fourth signalling group further comprises a second fluorescent group, the second fluorescent group is configured to be excited by chemiluminescence of the second chemiluminescent molecule.

19. The method according to any preceding claim, wherein the genetic display system is combined with a purification resin before step (a) such that the genetic display system is bound to the purification resin.

20. The method according to claim 19, wherein the bound genetic display system is further treated with a reducing agent before step (a).

21. The method according to claim 20, wherein the reducing agent is TCEP.

22. The method according to any of claims 19-21, wherein the genetic display system is eluted from the purification resin after step (b).

23. The method according to any preceding claim, wherein the genetic display system is phage display.

24. The method according to any preceding claim, wherein the peptide ligand includes at least one loop which comprises a sequence of amino acids subtended between two of the two or more amino acid residues.

25. The method according to any preceding claim, wherein the molecular scaffold is selected from the group of TBMB, TATA, TCAZ and TCCU.

26. The method according to any preceding claim, wherein the peptide ligand is a single clone or a library of peptide ligands.


SEQUENCE LISTING (31 sequences)

SEQ ID NO: 1 - protein, 15 aa, Artificial Sequence
  source: /note=“Description of Artificial Sequence Synthetic peptide”
CPYSWETCLFGDYRC

SEQ ID NO: 2 - protein, 15 aa, Artificial Sequence
  source: /note=“Description of Artificial Sequence Synthetic peptide”
CPLVNPLCLTSGWKC

SEQ ID NO: 3 - protein, 15 aa, Artificial Sequence
  source: /note=“Description of Artificial Sequence Synthetic peptide”
CGHVAPWCWRTNHDC

SEQ ID NO: 4 - protein, 14 aa, Artificial Sequence
  source: /note=“Description of Artificial Sequence Synthetic peptide”
CYNEFGCEDFYDIC

SEQ ID NO: 5 - protein, 13 aa, Artificial Sequence
  source: /note=“Description of Artificial Sequence Synthetic peptide”
SGTGAASTGGATC

SEQ ID NO: 6 - protein, 15 aa, Artificial Sequence
  source: /note=“Description of Artificial Sequence Synthetic peptide”
CPYSWETSLFGDYRS

SEQ ID NO: 7 - protein, 15 aa, Artificial Sequence
  source: /note=“Description of Artificial Sequence Synthetic peptide”
CPYSWETCLFGDYRS

SEQ ID NO: 8 - protein, 15 aa, Artificial Sequence
  source: /note=“Description of Artificial Sequence Synthetic peptide”
SPYSWETSLFGDYRS

SEQ ID NO: 9 - protein, 11 aa, Artificial Sequence
  source: /note=“Description of Artificial Sequence Synthetic peptide”
CQGMPCPRLPC

SEQ ID NO: 10 - protein, 11 aa, Artificial Sequence
  source: /note=“Description of Artificial Sequence Synthetic peptide”
CALFICWMMLC

SEQ ID NO: 11 - protein, 11 aa, Artificial Sequence
  source: /note=“Description of Artificial Sequence Synthetic peptide”
CDPKLCNQVMC

SEQ ID NO: 12 - protein, 11 aa, Artificial Sequence
  source: /note=“Description of Artificial Sequence Synthetic peptide”
CSGSSCLQREC

SEQ ID NO: 13 - protein, 11 aa, Artificial Sequence
  source: /note=“Description of Artificial Sequence Synthetic peptide”
CSRSKCQHLKC

SEQ ID NO: 14 - protein, 11 aa, Artificial Sequence
  source: /note=“Description of Artificial Sequence Synthetic peptide”
CTRPPCILSSC

SEQ ID NO: 15 - protein, 11 aa, Artificial Sequence
  source: /note=“Description of Artificial Sequence Synthetic peptide”
CVDEWCDVDYC

SEQ ID NO: 16 - protein, 9 aa, Artificial Sequence
  source: /note=“Description of Artificial Sequence Synthetic peptide”
CKSGCNMMC

SEQ ID NO: 17 - protein, 9 aa, Artificial Sequence
  source: /note=“Description of Artificial Sequence Synthetic peptide”
CLQKCLKSC

SEQ ID NO: 18 - protein, 9 aa, Artificial Sequence
  source: /note=“Description of Artificial Sequence Synthetic peptide”
CLRTCSSNC

SEQ ID NO: 19 - protein, 9 aa, Artificial Sequence
  source: /note=“Description of Artificial Sequence Synthetic peptide”
CRLLCLSQC

SEQ ID NO: 20 - protein, 9 aa, Artificial Sequence
  source: /note=“Description of Artificial Sequence Synthetic peptide”
CRPPCAPRC

SEQ ID NO: 21 - protein, 15 aa, Artificial Sequence
  source: /note=“Description of Artificial Sequence Synthetic peptide”
YNTMVVDYQPVGKKC

SEQ ID NO: 22 - protein, 15 aa, Artificial Sequence
  source: /note=“Description of Artificial Sequence Synthetic peptide”
CAKLALPYNFTTAGY

SEQ ID NO: 23 - protein, 15 aa, Artificial Sequence
  source: /note=“Description of Artificial Sequence Synthetic peptide”
CATTAVPYNFHTHTY

SEQ ID NO: 24 - protein, 15 aa, Artificial Sequence
  source: /note=“Description of Artificial Sequence Synthetic peptide”
YESVPMLYHDENSEC

SEQ ID NO: 25 - protein, 15 aa, Artificial Sequence
  source: /note=“Description of Artificial Sequence Synthetic peptide”
CKANRVPYNVNPDKC

SEQ ID NO: 26 - protein, 15 aa, Artificial Sequence
  source: /note=“Description of Artificial Sequence Synthetic peptide”
CAMAPQTYQGVLSSY

SEQ ID NO: 27 - protein, 15 aa, Artificial Sequence
  source: /note=“Description of Artificial Sequence Synthetic peptide”
YHKRMPKYNKMELRC

SEQ ID NO: 28 - protein, 15 aa, Artificial Sequence
  source: /note=“Description of Artificial Sequence Synthetic peptide”
CPMVNPLCLHPGWIC

SEQ ID NO: 29 - protein, 11 aa, Artificial Sequence
  source: /note=“Description of Artificial Sequence Synthetic peptide”
GCGSGCGSGCG

SEQ ID NO: 30 - protein, 35 aa, Artificial Sequence
  source: /note=“Description of Artificial Sequence Synthetic polypeptide”
  MOD_RES (3)..(17): Any amino acid
  SITE (3)..(17): /note=“This region may encompass 2-15 residues”
  MOD_RES (19)..(33): Any amino acid
  SITE (19)..(33): /note=“This region may encompass 2-15 residues”
  source: /note=“See specification as filed for detailed description of substitutions and preferred embodiments”
ACXXXXXXXXXXXXXXXCXXXXXXXXXXXXXXXCG

SEQ ID NO: 31 - protein, 11 aa, Artificial Sequence
  source: /note=“Description of Artificial Sequence Synthetic peptide”
ACGSGCGSGCG